SEQUENCE LISTING (4 sequences)

SEQ ID NO: 1 - DNA, 20 bp, Artificial Sequence
  Synthetic
  misc_feature: PD-L1 Forward Primer
atgccccatacaacaaaatc

SEQ ID NO: 2 - DNA, 21 bp, Artificial Sequence
  Synthetic
  misc_feature: PD-L1 Reverse Primer
gacatgtcagttcatgttcag

SEQ ID NO: 3 - DNA, 15 bp, Artificial Sequence
  Synthetic
  misc_feature: GAPDH Forward Primer
cttttgcgtcgccag

SEQ ID NO: 4 - DNA, 20 bp, Artificial Sequence
  Synthetic
  misc_feature: GAPDH Reverse Primer
ttgatggcaacaatatccac

(19) United States
(12) Patent Application Publication
(10) Pub. No.: US 2022/0347267 A1
(43) Pub. Date: Nov. 3, 2022

(54) METHODS TO IMPROVE PATIENT RESPONSE TO IMMUNE CHECKPOINT INHIBITORS AND FUNCTIONAL TESTS TO PREDICT RESPONSE

(71) Applicant: Biomarker Strategies, LLC, Rockville, MD (US)

(72) Inventors: Greg P. Bertenshaw, Rockville, MD (US); Marika Magdelena Smith, Rockville, MD (US); Meena Chandok, Rockville, MD (US); Senthamil Selvan, Rockville, MD (US); William Murphy, Rockville, MD (US); Palak Parekh, Rockville, MD (US)

(21) Appl. No.: 17/761,077

(22) PCT Filed: Sep. 17, 2020
(86) PCT No.: PCT/US2020/051197

§ 371 Date: Mar. 16, 2022

Related U.S. Application Data
(60) Provisional application No. 62/902,302, filed on Sep. 18, 2019.

(51) Int. Cl. A61K 38/19; G01N 33/574; A61P 37/02

(52) U.S. Cl. CPC A61K 38/19; G01N 33/57484; A61P 37/02

(57) ABSTRACT

The present invention provides methods to improve a cancer patient's response to immune checkpoint inhibitor (ICI) therapy, and functional test to predict likelihood of response. Specifically, the invention provides methods to improve patient's response to immune checkpoint inhibitors using modulators and to predict response to the combination of immune checkpoint inhibitors and modulators.

### CROSS-REFERENCE TO RELATED APPLICATIONS

This application claims benefit of priority under 35 U.S.C. § 119(e) of U.S. Ser. No. 62/902,302, filed Sep. 18, 2019, the entire contents of which is incorporated herein by reference in its entirety.

### GOVERNMENT SUPPORT STATEMENT

This invention was made with government support under Contract Nos: HHSN261201600006C and 75N91019C00022 awarded by the National Institutes of Health. The government has certain rights in the invention.

### INCORPORATION OF SEQUENCE LISTING

The material in the accompanying sequence listing is herein incorporated by reference into this application. The accompanying sequence listing file, name BMS1150_IWO_Sequence_Listing.txt, was created on Sep. 15, 2020, and is 2 kb. The file can be assessed by using Microsoft word on a computer that uses Windows OS.

### BACKGROUND OF THE INVENTION

### Field of the Invention

This invention is directed to methods to modulate the baseline level and/or forms of immune checkpoint proteins including PD-1 ligands (PD-L1 and PD-L2) and others.

### Background Information

By modulating the levels and/or forms, a more desirable clinical outcome can be enabled in patients who may not respond optimally to immune checkpoint inhibitors (ICI). The accompanying ex vivo live-cell test identifies which modulator(s) is best suited to provide an optimal response to respond to ICI. The test can also be used to identify and/or monitor patients who may respond to ICI alone, or in combination with other therapies but who may have otherwise been classified as negative for biomarkers using other diagnostic tests.

Immune evasion is a hallmark of cancer in which the immune system is unable to mount an effective antitumor response. Recently, there have been significant advances in a class of ICI drugs, which re-engage the immune system to kill tumors. These drugs have been shown to yield durable responses in some patients with some key opinion leaders projecting survival in a subset of patients beyond 10 years. Specifically, PD-1 and PD-1 ligand (PD-L1 and PD-L2) targeted-ICI block the interaction of these key regulatory checkpoint proteins and allow the immune system to re-engage the tumor.

Despite the ability of ICI to generate robust, durable responses in some patients, many patients do not respond and there is a critical lack of: (1) Improving response rates; and (2) Clinically useful tests that can accurately identify, monitor, prognose and predict responses to ICI therapies.

The current invention relates to changing the response rate of ICI therapy through changing the abundance or state of ICI targets using modulators.

The current invention also relates to changing the response rate of ICI therapy through generation of a novel form of PD-L1 (p-Tyr-PD-L1). New tools (e.g. therapeutic antibodies) for the newly identified form (p-Tyr-PD-L1) can be used to increase the antitumor immunity, drug efficacy in monotherapy or combination therapies.

The abundance of PD-L1 expressed by a tumor is an established predictive marker for response to PD-1/PD-L1 ICI therapies. Traditional companion diagnostic (CDx) and complementary diagnostic (CoDx) tests for this biomarker rely on measuring PD-L1 by immunohistochemistry (IHC) in a cross section of tumor tissue biopsy obtained pre-ICI treatment and then categorizing patients as PD-L1 biomarker positive (+) or negative (−) based the number of cells staining positive for that biomarker. Patients categorized as biomarker positive are considered likely to respond to therapy and most go on to receive ICI therapy. Many biomarker positive patients do not respond well to therapy. Those categorized as biomarker negative may be considered not likely to respond but are often offered therapy. In many cases, these biomarker negative patients respond to ICI therapy.

This highlights the clear need for more accurate diagnostic tests that can better identify, predict and monitor patients who are likely to respond to ICI therapy, in particular the sub-group of patients who are biomarker negative by traditional IHC tests looking at basal level but who ultimately would be identified as biomarker positive.

Additionally, ICI monotherapies and combination therapies have the potential to cause severe, life-threatening side effects including but not limited to hyper-progressive disease (HPD). Therefore, it is important that any diagnostic test for these drugs also be able to help identify, monitor and predict those patients who are not expected to respond to therapies and in this way spare them from HPD and other unnecessary complications.

To address this critical need, we have developed a molecular test using live tumor cells or others cells within clinical tumor tissue sample that will be better able to identify, predict and monitor an individual's response to ICI monotherapies and combination therapies and that will enable oncologists to better tailor therapy regimes to the individual patient.

### SUMMARY OF THE INVENTION

The present invention is based on the seminal discovery that modulators such as IFNγ, TNFα and others can be used to improve a cancer patient's response to immune checkpoint therapy, and functional test to predict likelihood of response, and specifically to improve patient's response to immune checkpoint inhibitors.

In one embodiment, the invention provides a method to alter the level, form, and localization of immune checkpoint proteins using a modulator in a biological sample or subject to produce a more robust response to ICI and/or to identify, monitor, prognose, and predict responsiveness to an ICI monotherapy regime and/or combination regimes including: exposing a biological sample or subject which would otherwise not optimally respond to immune checkpoint inhibitors to the modulator to produce a robust response; and/or measuring static biomarker(s) and/or determining the difference between dynamic biomarker(s) in an unmodulated portion of the sample and after contacting one or more portions of the sample with one or more modulators ex vivo, prior to, during, simultaneously with, throughout, or following the administration of ICI monotherapies and/or combination therapies; wherein the difference in the basal and modulated biomarker(s) and the modulated portion(s) of the sample is expressed as a value which is predictive of a positive or negative response to ICI monotherapies and/or combination therapies.

In one aspect, the sample is from a subject with cancer. In various aspects, the cancer is selected from the group consisting of colorectal, esophageal, stomach, lung, mesothelioma, prostate, uterine, breast, skin, endocrine, melanoma, urinary, pancreas, ovarian, cervical, head and neck, liver, bone, biliary tract, small intestine, hematopoietic/blood cancers (myeloma, leukemia, and lymphoma), vaginal, testicular, anal, kidney, brain, eye cancer, leukemia, lymphoma, soft tissue, melanoma, mixed types, and metastases thereof. In other aspects, the sample is from single or multiple tumor tissue of an unknown primary or any type of tumor. In many aspects, the tumor sample is from a solid tumor.

In another aspect, the tumor sample is obtained by fine needle aspiration, core biopsy, collecting circulating tumor cells, surgical excision, or other tumor sample acquisition method.

In other aspects, the method further includes stratification of patients on their predicted responsiveness to a therapeutic agent or therapeutic regimen and providing a positive or negative treatment value which corresponds to positive or negative clinical outcome, respectively.

In some aspects, the method is further used to identify, monitor, predict the existence of and characterize functional sub-populations (i.e. heterogeneity) of cancer cells, which can be used to predict a positive or negative response to ICI monotherapies and/or combination therapies; and the identification and characterization of functional sub-populations can be used to predict and identify the mechanisms of innate and acquired resistance to ICI monotherapies and/or combination therapies. This information can be used to inform the creation and tailoring of ICI monotherapies and/or combination therapies regimens to the individual patient.

In one aspect, the biological sample is a cancer cell or a cancer cell subpopulation, including but not limited to cancer stem cells, wherein the cancer stem cell expresses or one more of CD133, CD44, ABCG2, and/or ALDH1A1 and/or does not stain using Hoechst. In another aspect, the biological sample is immune cells or immune cell subpopulation, including but not limited to T cells, CD8+ T cells, exhausted T cells, active T cells, B cells, NK cells, dendritic cells, myeloid derived suppressor cells, macrophages, granulocytes, and mast cells. In yet another aspect, the modulator is a biologic agent, biosimilar, a derivative thereof, a mutant thereof, a peptide thereof, a fragment thereof, an analog thereof or a mimetic thereof; and the derivative, mutant, peptide, fragment, analog or mimetic of the modulator enhances or decreases half-life and/or target binding, and has a similar effect as the intact modulator.

In some aspects, the modulator is an interferon; an interleukin; FGF, EGF, VEGF, prostaglandin E2 (PGE2), prostacyclin (PGI2), prostaglandin D2 (PGD2) and prostaglandin F2α (PGF2α), IDO, GM-CSF, TNF-alpha, TGF-beta, a derivative thereof, a mutant thereof, a peptide thereof, a fragment thereof, an analog thereof or a mimetic thereof; and the derivative, mutant, peptide, fragment, analog or mimetic of the modulator enhances or decreases half-life and/or target binding, and has a similar effect as the intact modulator.

In one aspect, the biological sample/cancer subject is treated in a manner to alter conditions selected from the group consisting of the abundance of proteins, carbohydrates, lipids, molecules found within fetal bovine serum, as well as growth factors, hypoxia, oxidative stress, and physical exercise/stress, or by using a patients' “milieu” from a tumor biopsy optionally consisting of various cell types selected from the group consisting of fibroblasts, inflammatory and immune cells, and soluble factors derived from tumor and associated cells; or using the patients' own immune cells selected from those derived from tumors (e.g. T cells, B cells, NK (Natural Killer) cells, macrophages and dendritic cells, myeloid derived suppressor cells and granulocytes) or peripheral blood immune cells (e.g. T cell, B cells, NK cells, monocytes, dendritic cells, tumor cells, granulocytes, NK cells).

In some aspects, multiple modulators are added together, sequentially or alternately.

In one aspect, the modulator is reactive oxygen species or a free radical molecule, a radiation therapy selected from the group consisting of x-rays, gamma rays, and charged particles. In another aspect, the modulator is a phenothiazine. in one aspect, the phenothiazine is prochlorperazine. In other aspects, the modulator is a chemotherapeutic agent. In various aspects, the chemotherapeutic agent is an alkylating agent, a plant alkaloid, an antitumor antibiotic, an antimetabolite, a topoisomerase inhibitor, a molecularly targeted agent, selected from the group consisting of an inhibitor of EGFR, ALK, VEGF family KIT, HER2, CDK4 and CDK6, BRAF, PARP, JAK family, mTOR, MEK, ERK, PDGFR RAF, RET, MET, ROS1, PIGF, PTCH, Smoothened, RANKL, B4GALNT1, STAT, HDAC, BET, NTRK, Bcl-2 ATM, ATR, A2AR, WEE1, FGFR, and an autophagy influencer.

In one aspect, the biomarker is measured on tumor cells, stromal cells (e.g. fibroblasts) or immune cells (T cells, B cells, NK cells, dendritic cells, myeloid derived suppressor cells, macrophages, granulocytes, and mast cells) or subpopulation thereof. In some aspects, the tumor cell subpopulation includes a cancer stem cell that expresses one or more of CD133, CD44, ABCG2, and/or ALDH1A1. In other aspects, the biomarker is the localization, and/or level, and/or state of a molecule or molecules. In various aspects, the molecule(s) being measured is a protein or a nucleic acid; the state of the molecule(s) being measured is phosphorylation, acylation, alkylation, amidation, glypiation, glycation, glycosylation, ubiquitination, degradation product(s), truncation, mutation status, or binding of the molecule(s) to promoters that induce PD-1 ligand expression including GAS (Gamma Activated Sequence) promoter/enhancer regions and the promoter of PD-1 ligand genes; methylation status and chromatin modification of the promoter/enhancer region and the status of SWI/SNF complexes; the localization of the molecule(s) being measured is extracellular or cellular, wherein cellular localization includes intracellular, compartmentalized (e.g. Golgi, endoplasmic reticulum, lysosomal, endosomal, exosomal, mitochondrial, vacuole, and cytosolic), nuclear and nucleoli, or membrane (e.g. plasma, nuclear and other organelle membranes) bound including measurements of endocytosis and exocytosis.

In many aspects, the molecule(s) being measured is PD-1 or a PD-1 ligand (PD-L1 or PD-L2), AZAR, CD40, CD40L, galectin-9, IDO1, CTLA-4, KIRs, 4-1BB, GITR, T cell immunoglobulin mucin 3 (TIM-3), LAG-3 (CD223), Killer immunoglobulin-like receptors (KIRs), VISTA, Adenosine, MHC I and MHC II; the molecule(s) being measured is in a pathway that influences PD-1 ligand expression including the immediate target or downstream target of an Interleukin; is FGF, EGF, VEGF, prostaglandins (e.g. prostaglandin (PG) E2 (PGE2), prostacyclin (PGI2), prostaglandin D2 (PGD2) and prostaglandin F2α (PGF2α)), IDO, or GM-CSF; is in TNF-alpha, TGF-beta, the IFN alpha, beta, and gamma response pathways; in the JAK/STAT pathway, the EGFR response pathway, the PI3K pathway, the MAPK pathway, and/or the mevalonate pathway.

In various aspects, the molecule(s) being measured is IFN-alpha, beta and/or gamma receptors; from the STAT family (Signal Transducer and Activator of Transcription); from the JAK family (Janus Kinase); from the IRF family (Interferon Regulatory Factors); EGFR; from the PI3K pathway; is PTEN, PI3K, PDK1, AKT, mTOR, S6, S6-Kinase, CREB, GSK3B, mTORC1 or mTORC2; from the MAPK pathway; from the mevalonate pathway; a receptor tyrosine kinase; a hypoxia inducible factor; an interleukin receptor; a marker of autophagy; or a direct or indirect target of interferon, interleukin or TNF receptors.

In many aspects, the biomarker being measured is the abundance of endosomes or exosomes, the contents of endosomes or exosomes.

In one aspect, the ICI response biomarkers are combined with tumor mutation burden. In another aspect, the ICI response biomarkers are combined with p53 mutation status. In some aspects, the ICI response biomarkers are combined with the abundance, phenotypic and functional activity (cell proliferation, survival, and cytotoxicity) of tumor-infiltrating immune cells (e.g. myeloid dendritic cells, macrophages, granulocytes, mast cells and tumor-infiltrating lymphocytes (TILs); T helper cells, T cytotoxic cells, T regulatory cells, B cells and NK cells); the ICI response biomarkers are combined with PD-1 baseline level to predict a positive response to anti-PD-1 therapy for patients with PD-L1 negative tumors; or the ICI response biomarkers are combined with markers of immune senescence (diminished cell proliferation, cytokine secretion (such as IFNγ, TNFα and IL-2), display of PD-1, Tim3 and LAGS), and lysosomal-associated membrane protein-1 (also referred to as CD107a) indicative of T cell impairment and immune escape.

In other aspects, the response biomarkers are combined with static expression levels of the biomarkers including PD-1, PD-1 ligand, AZAR, CD40, CD40L, galectin-9, IDO1, CTLA-4, KIRs, 4-1BB, GITR, T cell immunoglobulin mucin 3 (TIM-3), LAG-3 (CD223), Killer immunoglobulin-like receptors (KIRs), VISTA, Adenosine, MHC I and MHC II. In yet another aspect, the ICI response biomarkers are combined with the size of the MHC-bound peptides.

In one aspect, the molecules being measured are an Interleukin, FGF, EGF, VEGF, prostaglandins (e.g. prostaglandin (PG) E2 (PGE2), prostacyclin (PGI2), prostaglandin D2 (PGD2) and prostaglandin F2α (PGF2α)), IDO, GM-CSF, TNF-alpha, and TGF-beta, which are generated by the tumor cells or cells found in the tumor microenvironment.

In many aspects, the biomarker being measured is Toll-like receptor (TLR), NKG2D, B3NT3, or NFkBP65; SWI/SNF complex, BRG1, BAF180, CTCF, a miRNA selected from the group consisting of miRNA-570, miRNA-513 and miRNA-155.

In other aspects, the response biomarkers are combined with the patient race, gender and age or age-related biomarkers selected from the group consisting of follicle-stimulating hormone, biopsy type, tumor stage, tumor type and/or histological categorization (e.g. cell cycle status, cell type and differentiation status) of the sample.

In an additional aspect, the expression level of a biomarker is normalized against a normalization biomarker selected from the group consisting of Na/K-ATPase, HSP90, actin, GAPDH, vinculin, tubulin, and histone.

In some aspects, the biomarker(s) are measured using immunoassays, multiplexed assays, PCR, transcription factor assays, DNase hypersensitivity assays, nucleic acid or sequencing/mutation testing. In one aspect, the analytical technique is an immunoassay (e.g. western blot, dot blot, ELISA, immunohistochemistry, immunocytochemistry, immunofluorescence).

In yet another aspect, the biological sample is processed in part, or entirely, using one or more manual methods and/or automated systems.

In some aspects, the biomarker or panel of biomarkers selected from the group consisting of PD-1, PD-L1, PD-L2, AZAR, CD40, CD40L, galectin-9, IDO1, CTLA-4, KIRs, 4-1BB, GITR, TIM-3, LAG-3, lysosomal-associated membrane protein-1, KIRs, VISTA, Adenosine, MHC I, MHC II, STAT-1, STAT-2, STAT-3, STAT-4, JAK-1, JAK-2, IRF-9, EGFR, PTEN, PI3K, PDK1, AKT, mTOR, S6, S6-Kinase, CREB, GSK3B, mTORC1, mTORC2, Src, Fak, Ras, Raf, Mek, Erk, CREB, Sos-1, SHC, NFkB, cMyc, ELK-1, Tristetraprolin, c-Fos, c-Jun, RET, EGFR, MET, KIT, ALK, HER2, ROS1, JAK2, VEGFR, PDGFR, Hif-1a, Hif-2a, GP130, IL1R, TNFR, IL2Ra/b/g, TRAF2, TRAF-5, TRAF-6, I-Kappa B, MyD88, IRAK1, IRAK2, IRAK3, IRAK4, MKK, IKB, IKK, NFATc1, p38, p48, JNK, AP1, IRS, SHC, GRB2, SOS, Toll-like receptor (TLR), NKG2D, B3NT3, NFkBP65, SWI/SNF complex, BRG1, BAF180, CTCF, miRNA-570, miRNA-513, miRNA-155, IFN-α, IFN-β, or IFN-γ, IL-1, IL-2, IL-4, IL-6, IL-7, IL-8, IL-10, IL-12, IL-15, IL-17, IL-18, IL-21, IL-22, IL-23, IL-27, IL-35, FGF, EGF, VEGF, prostaglandin E2, prostacyclin, prostaglandin D2, prostaglandin F2α, IDO, GM-CSF, TNF-alpha, TGF-beta, tumor mutation burden, p53 mutation status, the abundance, phenotypic and functional activity, including cell proliferation, survival, and cytotoxicity, of tumor-infiltrating immune cells selected from the group consisting of myeloid dendritic cells, macrophages, granulocytes, mast cells and tumor-infiltrating lymphocytes (TILs); T helper cells, T cytotoxic cells, T regulatory cells, B cells and NK cells, patient race, gender and age or age-related biomarkers selected from the group consisting of follicle-stimulating hormone, biopsy type, tumor stage, tumor type and/or histological categorization including cell cycle status, cell type and differentiation status of the sample, actin, GAPDH, vinculin, tubulin, and histone is used to predict the likelihood of response including detecting the expression levels of biomarkers in a specimen as compared with a non-responsive group and a responsive group of samples; and the expression levels of the biomarkers are evaluated by applying a statistical method selected from the group consisting of receiver operating characteristic (ROC) curve cut point analysis, regression analysis, discriminant analysis, classification tree analysis, random forests, support vector machine, OneR, kNN and heuristic naive Bayes analysis, neural nets and variants thereof.

In other aspects, the correlating is performed by a software classification algorithm.

In one aspect, the modulator includes one or more therapeutic PD-1 or PD-L1 antibodies selected from the group consisting pembrolizumab, nivolumab, cemiplimab, atezolizumab, avelumab, and durvalumab. In some aspects, the therapeutic antibody is used in the detection technique, for example, it is conjugated to a detection molecule selected from the group consisting of fluorescein isothiocyanate (FITC), Qdot 655, horseradish peroxidase (HRP), and alkaline phosphatase (AP). In one aspect, multiple therapeutic PD-1 or PD-L1 antibodies are used as a modulator on a single sample. In some aspects, each therapeutic antibody is conjugated to a unique detection molecule to compare ability to bind the target, where the detection molecule is selected from the group consisting of FITC, tetramethylrhodamine (TRITC), Qdot 655, HRP, AP, and 3,3′-Diaminobenzi dine (DAB). In one aspect, the biomarker is the ability for PD-1 or PD-L1 protein to bind to PD-L1 or PD-1, respectively. In other aspects, the PD-1 or PD-L1 protein is conjugated to a single detection molecule selected from the group consisting of FITC, TRITC, Qdot 655, HRP, DAB, and AP. In other aspects, the biomarker is the intensity of detection molecules in the sample.

### BRIEF DESCRIPTION OF THE DRAWINGS

FIG. 1 illustrates a proposed role of PTM in PD-L1 regulation.

FIGS. 2A-2BC show the effects of TNFα and IFNγ modulators on the levels of PD-L1 in lung cancer cells H2170. FIG. 2A shows the PD-L1 immunostaining of the cells after treatment; FIG. 2B is a graph bar illustrating PD-L1 quantification; and FIG. 2C is a magnified view of the cells after treatment with TNFα.

FIGS. 3A-3C show the effects of TNFα and IFNγ modulators on the level of PD-L1 in lung cancer cells H2126. FIG. 3A shows the PD-L1 immunostaining of the cells after treatment; FIG. 3B is a graph bar illustrating PD-L1 quantification; and FIG. 3C is a magnified view of the cells after treatment with TNFα.

FIGS. 4A-4B show the effects of TNFα and IFNγ modulators on the level of PD-L1 in lung cancer cells HCC827. FIG. 4A shows the PD-L1 immunostaining of the cells after treatment; FIG. 4B is a graph bar illustrating PD-L1 quantification.

FIGS. 5A-5B show the effects of TNFα (T60) and IFNγ (I60) modulators on the level of PD-L1 in melanoma cells A375 within 60 minutes. FIG. 5A shows the PD-L1 immunostaining of the cells after treatment; FIG. 5B is a graph bar illustrating PD-L1 quantification.

FIG. 6 shows the PD-L1 the effects of TNFα and IFNγ modulators on the level of PD-L1 in a lung cancer clinical sample evaluated by immunohistochemistry.

FIGS. 7A-7C illustrate the conversion effect of TNFα modulator on immunotherapy non-responder cancer cells. FIG. 7A illustrates the how PTM converts immunotherapy non-responder sample into immunotherapy responder; FIG. 7B shows the PD-L1 immunostaining of immunotherapy non-responder lung cancer cells (H2170) after treatment with TNFα; FIG. 7C shows the PD-L1 immunostaining of a lung cancer clinical sample after treatment with TNFα.

FIGS. 8A-8C illustrate the changes in PD-L1 levels via modulator IFNγ in lung cancer PDX sample. FIG. 8A illustrates the viability of the cells assessed by flow cytometry; FIG. 8B illustrates an immunostaining of the cells; FIG. 8C illustrates PD-L1 levels before and after treatment with IFNγ, assessed by flow cytometry.

FIGS. 9A-9E show the changes in PD-L1 level in tumor and immune cells by modulator IFNγ in lung cancer clinical sample. FIG. 9A shows immunostaining of immune and tumor cells in a clinical sample; FIG. 9B illustrates the number of CD45+ cells before and after IFNγ treatment; FIG. 9C illustrates immune and tumor cells viability, by flow cytometry; FIG. 9D illustrates PD-L1 levels in tumor cells before and after treatment with IFNγ, by flow cytometry; and FIG. 9E illustrates PD-L1 levels in immune cells before and after treatment with IFNγ, by flow cytometry.

FIG. 10 shows changes in PD-L1 and pTyr levels via modulators IFNγ and TNFα in HCC827 lung cancer cells by immunofluorescence.

FIG. 11 is a graph bar illustrating the quantification of pPD-L1 levels after modulators (IFNγ and TNFα) treatment in lung cancer cells.

FIGS. 12A-12B illustrate the changes in PD-L1 levels after immunoprecipitation of by pTyr. FIG. 12A shows results obtained in lung cancer HCC827; and FIG. 12B shows results obtained in melanoma cell line A375.

FIG. 13 shows the effect of TNFα (T) and IFNγ (I) modulators on PD-L1 levels in isolated membrane fractions in melanoma cell line A375.

FIGS. 14A-14B show the effects of modulator IFNγ on PD-L1 and pTyr levels in lung cancer cell line HCC827 measured by flow cytometry. FIG. 14A illustrates PD-L1 levels; FIG. 14B illustrates pTyr levels.

FIGS. 15A-15B show the effect of IFNγ and TNFα modulators on PD-L1 and pTyr levels in melanoma cells A375. FIG. 15A shows the levels measured by flow cytometry; FIG. 15B illustrate the post-translational changes in different forms of PDL-1 proteins after modulator(s) treatment, evaluated by western blot.

FIG. 16 illustrate the changes in total Tyr phosphorylation level by phosphatase inhibitor in lung cancer cell line (H292).

FIG. 17 shows PD-L1 levels in H292 NSCLC cells in response to various doses of a phosphatase inhibitor by western blot.

FIGS. 18A-18B illustrate the changes in viability status after phosphatase inhibitor treatment in lung cancer cell line (H292). FIG. 18A shows cell viability by immunofluorescence;

FIG. 18B is a graph bar illustrating cell viability quantification.

FIGS. 19A-19B illustrate tyrosine phosphorylation residues in PD-L1. FIG. 19A shows PD-L1 amino acid sequence; FIG. 19B shows the putative phosphorylated tyrosine positions.

FIG. 20 illustrates distinct expresser types in various cancer types. Each paired sample set represents a unique patient derived cell line of a specific cancer type; and the effect of a treatment with IFNγ for 24h on PD-L1 modulation was evaluated by western blot.

FIG. 21 illustrates distinct expresser type in NSCLC clinical samples. Each paired sample set (A, B, and C) represents a unique NSCLC clinical sample; and the effect of a treatment with IFNγ for 24h on PD-L1 modulation was evaluated by western blot.

FIGS. 22A-22B represent distinct effects of therapeutic modulators on PD-L1 expression. FIG. 22A illustrates the effect of Pemetrexed (PMX); FIG. 22B illustrates the effects of Carboplatin (CARBO).

FIG. 23 illustrates the three type of cancer cell response to a treatment with a therapeutic modulator Pemetrexed (PMX) or Carboplatin (CARBO)); increased responder (left panel). Decreased responder (middle) or non-responder (right panel).

FIGS. 24A-24B illustrates various biomarkers modulation in response to IFNγ modulation treatment. FIG. 24A illustrates the modulation of P-STAT1, IRF-1, PD-L1, and PD-L2 in H1975 and H2126 cancer cells; FIG. 24B illustrates the modulation of P-STAT3, P-STAT1, IRF-1, IRF-9, and PD-L1 in SKLU1, H23, H1703, H2170, H1650 and SKMES1 cancer cells.

FIG. 25 is a schematic representation of the protocol followed to treat cells with a modulator, collect the cells and prepare cell lysate at different time points.

FIGS. 26A-26F illustrate the modulation of IFNΓR1, pJAK2, pSTAT1, pSTAT3, IRF-1, IRF-9, CDK5, DNMT1, PD-L1, and PD-L2 biomarkers in response to IFNγ modulation treatment, at 30 min and 24 hours post-treatment. FIG. 26A illustrates the modulation in H2170 cancer cells; FIG. 26B illustrates the modulation in H1703 cancer cells; FIG. 26C illustrates the modulation in H23 cancer cells; FIG. 26D illustrates the modulation in SKLU1 cancer cells;

FIG. 26E illustrates the modulation on H1650 cancer cells; and FIG. 26F illustrates the modulation in HCC827 cancer cells.

FIG. 27 illustrates an example of a predictive test matrix using TMB, baseline PD-L1 IHC and PathMAP approaches; (−) indicates individual considered biomarker negative by that particular test, while (+) indicates biomarker positive.

FIG. 28 illustrates a workflow for the quantitative automated analysis of immunocytochemical (ICC) images.

FIG. 29 illustrates immunocytochemical images of IFNγ induced PD-L1 expression in NSCLC SKMES1 cells. Membrane (arrows upper panel) and cytoplasmic (arrows in lower panels) staining are detected.

FIG. 30 illustrates examples of immunocytochemical images of weak and strong IFNγ induced PD-L1 expression.

FIG. 31 illustrates examples of PD-L1 negative cells (white arrows) and varying degrees of positive (dark arrows) tumor-like cells (NSCLC clinical tissue samples).

FIGS. 32A-32B illustrate the analysis of SKMES1 cell line treated with interferon-g before and after quantitative ICC analysis. FIG. 32A illustrates examples of immunocytochemical images of SKMES1 cell line treated with IFNγ before and after quantitative ICC analysis. FIG. 32B illustrates the proportional intensity of PDL1 shown as a histogram in treated and untreated sample.

FIGS. 33A-33E illustrates the flow cytometry analysis and gating strategy to analyze PDL1 expression. FIG. 33A illustrate the scatter gate applied on all events captured. FIG. 33B illustrates doublets exclusion by application of a singlet gate on the scatter population. FIG. 33C illustrates the PD-L1 gate placed adjacent to the highest PD-L1 signal of isotype control sample.

FIG. 33D illustrates the population hierarchy of four different populations. FIG. 33E shows a statistics table showing the number of events, percentage of parent population, percentage of total population and the mean and the median PD-L1 signal in each population.

FIGS. 34A-34H illustrate PD-L1 expression in treated and untreated cells and statistics table for each sample. FIG. 34A shows PD-L1 expression in treated SKMES1 cells.

FIG. 34B shows PD-L1 expression in untreated SKMES1 cells. FIG. 34C shows PD-L1 expression in treated H460 cells. FIG. 34D shows PD-L1 expression in untreated H460 cells.

FIG. 34E shows PD-L1 expression in treated H1299 cells. FIG. 34F shows PD-L1 expression in untreated H1299 cells. FIG. 34G shows PD-L1 expression in treated H1650 cells. FIG. 34H shows PD-L1 expression in untreated H1650 cells.

FIGS. 35A-35B illustrate total PD-L1 expression in four cell lines upon stimulation with IFNγ. FIG. 35A shows representative immunofluorescent images of the cells. FIG. 35B is a bar graph representing PD-L1 quantification.

FIGS. 36A-36B illustrate representative immunoblots of examples of responses as determined by western blot. FIG. 36A shows expression responses of intact PD-L1 to various modulators. FIG. 36B shows the detection of secreted PD-L1.

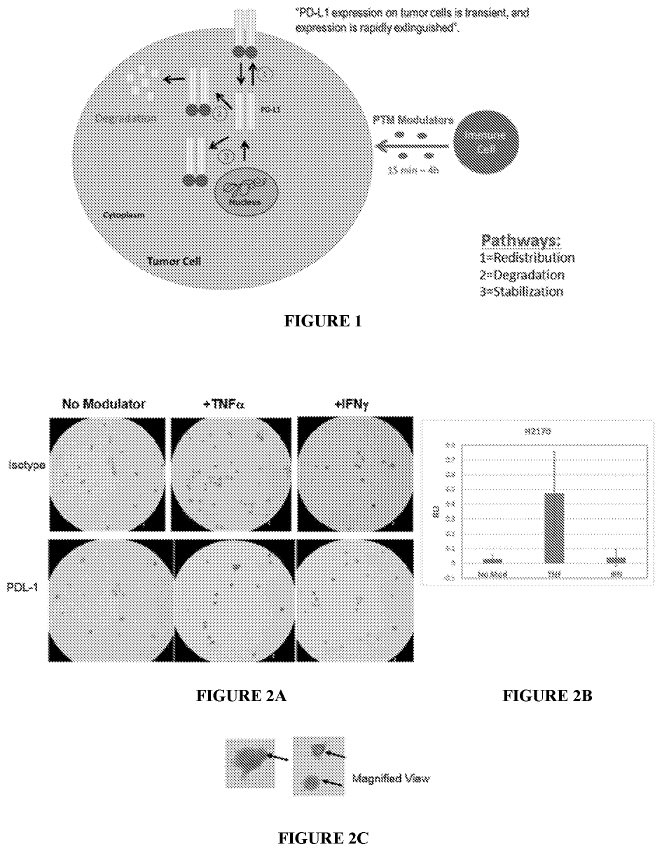

### DETAILED DESCRIPTION ON THE INVENTION

The present invention is based on the seminal discovery that modulators can be used to improve a cancer patient's response to immune checkpoint therapy and the dynamic changes to key proteins can be detected to predict the outcome to therapy.

Before the present compositions and methods are described, it is to be understood that this invention is not limited to particular compositions, methods, and experimental conditions described, as such compositions, methods, and conditions may vary. It is also to be understood that the terminology used herein is for purposes of describing particular embodiments only, and is not intended to be limiting, since the scope of the present invention will be limited only in the appended claims.

As used in this specification and the appended claims, the singular forms “a”, “an”, and “the” include plural references unless the context clearly dictates otherwise. Thus, for example, references to “the method” includes one or more methods, and/or steps of the type described herein which will become apparent to those persons skilled in the art upon reading this disclosure and so forth.

All publications, patents, and patent applications mentioned in this specification are herein incorporated by reference to the same extent as if each individual publication, patent, or patent application was specifically and individually indicated to be incorporated by reference.

Unless defined otherwise, all technical and scientific terms used herein have the same meaning as commonly understood by one of ordinary skill in the art to which this invention belongs. Although any methods and materials similar or equivalent to those described herein can be used in the practice or testing of the invention, it will be understood that modifications and variations are encompassed within the spirit and scope of the instant disclosure. The preferred methods and materials are now described.

In one embodiment, the invention provides a method to alter the level, form, and localization of immune checkpoint proteins using a modulator in a biological sample or subject to produce a more robust response to immune checkpoint inhibitor (ICI) and/or to identify, monitor, prognose, and predict responsiveness to an ICI monotherapy regime and/or combination regimes including: exposing a biological sample or subject which would otherwise not optimally respond to immune checkpoint inhibitors to the modulator to produce a robust response; and/or measuring static biomarker(s) and/or determining the difference between dynamic biomarker(s) in an unmodulated portion of the sample and after contacting one or more portions of the sample with one or more modulators ex vivo, prior to, during, simultaneously with, throughout, or following the administration of ICI monotherapies and/or combination therapies; wherein the difference in the basal and modulated biomarker(s) and the modulated portion(s) of the sample is expressed as a value which is predictive of a positive or negative response to ICI monotherapies and/or combination therapies.

ICI have been approved as both monotherapies and in combination with other therapies, including chemotherapeutic agents and molecularly targeted agents (MTAs) for the treatment of a wide variety of solid tumors. The ex vivo tests discussed within can broadly be applied to all ICI monotherapies and combination therapies being used to treat any type of tumor. Additionally, the ex vivo test can be applied to ICI in combination with various biologics and other modulators. As way of example, one ex vivo test probes the functional activity of regulatory pathway(s) to determine if a patient's tumor cells can innately (self-sufficient) or adaptively (external modulators; e.g. IFNγ) express PD-1 ligands (e.g. PD-L1). The ability to distinguish the expresser types (“non-expressers” and “innate expressers”) especially from “adaptive expressers” will help to stratify patients and help predict responses to checkpoint blockade therapy. Additionally, the new form of PD-L1 (p-Tyr-PD-L1) can be a therapeutic target itself or can be detected for predictive tests to stratify patients for providing optimal benefit over the existing PD-L1 therapy in use so far.

“Checkpoint inhibitor therapy” is a form of cancer treatment currently that uses immune checkpoints which affect immune system functioning. Immune checkpoints can be stimulatory or inhibitory. Tumors can use these checkpoints to protect themselves from immune system attacks. Checkpoint therapy can block inhibitory checkpoints, restoring immune system function. Checkpoint proteins include programmed cell death 1 protein (PDCD1, PD-1; also known as CD279) and its ligands, PD-1 ligand 1 (PD-L1, CD274) and PD-L2, as well as cytotoxic T-lymphocyte-associated protein 4 (CTLA-4), A2AR (Adenosine A2A receptor), B7-H3 (or CD276), B7-H4 (or VTCN1), BTLA (B and T Lymphocyte Attenuator, or CD272), IDO (Indoleamine 2,3-dioxygenase), KIR (Killer-cell Immunoglobulin-like Receptor), LAG3 (Lymphocyte Activation Gene-3), TIM-3 (T-cell Immunoglobulin domain and Mucin domain 3), and VISTA (V-domain Ig suppressor of T cell activation).

Programmed cell death protein 1, also known as PD-1 and CD279 (cluster of differentiation 279), is a cell surface receptor that plays an important role in down-regulating the immune system and promoting self-tolerance by suppressing T cell inflammatory activity. PD-1 is an immune checkpoint and guards against autoimmunity through a dual mechanism of promoting apoptosis (programmed cell death) in antigen-specific T-cells in lymph nodes while simultaneously reducing apoptosis in regulatory T cells (anti-inflammatory, suppressive T cells).

PD-1 has two ligands, PD-L1 and PD-L2, which are members of the B7 family. PD-L1 protein is upregulated on macrophages and dendritic cells (DC) in response to LPS and GM-CSF treatment, and on T cells and B cells upon TCR and B cell receptor signaling, whereas in resting mice, PD-L1 mRNA can be detected in the heart, lung, thymus, spleen, and kidney. PD-L1 is expressed on almost all murine tumor cell lines, including PA1 myeloma, P815 mastocytoma, and B16 melanoma upon treatment with IFN-γ. PD-L2 expression is more restricted and is expressed mainly by DCs and a few tumor lines.

The ex vivo test can be performed manually, or optionally performed using an automated system. Unlike traditional indirect approaches that measure static baseline levels of molecules in dead tissue, the current invention utilizes the patient's live tumor cells, and exposes them, ex vivo, to different modulators and measures the functional responses of the cells. The generated functional signaling profiles are used to predict whether an individual patient will respond to specific monotherapies or combinations of therapies in vivo. The approach can identify biomarker positive individuals, but more importantly, it can help identify individuals who are considered biomarker negative by other, more traditional diagnostic tests and who may have otherwise been considered unlikely to respond to therapies.

In the context of ICI therapies, patients whose initial biopsies do not express basal PD-L1 as measured using traditional IHC based tests are considered PD-L1 “biomarker negative”. This biomarker negative status indicates that they are considered unlikely to respond to PD-1/PD-L1 therapies and can be possibly excluded from taking these therapies that can have a significant impact on quality of life and survival. However, studies have shown that a reliable portion of the biomarker negative individuals ultimately respond to ICI monotherapies and combination therapies. In other instance, biomarker positive individuals are observed to be non-responder to ICI therapies. Our ex vivo test can identify a subset of these patients by determining if the ICI targets are correctly expressed (e.g. the form and localization of the target protein) and if the components of the microenvironment are suitable for a response.

The diagnostic test is intended to help with identifying, monitoring and predicting the therapeutic responses to ICI monotherapies and combination therapies in “biomarker negative” individuals determined by available conventional tests who ultimately respond to therapy.

One of the reasons why traditional IHC tests misidentify so many patients as being unlikely to respond is because PD-L1 expression is a highly dynamic process influenced by several internal and external factors including mutations and aberrant signaling pathways. The ex vivo test will allow an assessment of the dynamic expression of PD-1 ligands. Unlike the current invention, IHC tests cannot predict how these factors could influence PD-L1 expression. Notably, the current invention can directly assess the tumor-specific influence of different MTA therapies on biomarker expression when taken alongside ICI therapies.

In addition to traditional IHC testing for PD-L1 baseline expression, there is one other well established biomarker shown to predict responses to ICI therapies: Tumor Mutation Burden (TMB). TMB is a measurement of how many somatic mutations are present in the cancer's genome per mega base and is essentially a surrogate marker of tumor antigenicity. Importantly, this biomarker is reported to be to be independent and unrelated of PD-L1 expression status. TMB “high” individuals vs. low are approximately equally distributed equally among PD-L1 biomarker positive and negative patient populations. As such, IHC and TMB diagnostic information can be considered supplementary to our invention and can be mathematically combined with ex vivo outcomes to improve predictions.

In some aspects, the method described herein can be used to identify, monitor and predict the existence of one or more functionally distinct cell populations, which can be applied to identify, monitor and predict the existence of innate and acquired resistance mechanisms against ICI monotherapies and combination therapies in vivo.

In some aspects, the methods described herein can be used with any type of aggregated cells or tumor cells from single or multiple tumor tissues in a given patient. For example, they can test and process carcinomas or sarcomas. Example cancers that can be tested with the present methods include, but are not limited to, colon cancer, rectal cancer, pancreatic cancer, breast cancer, ovarian cancer, prostate cancer, squamous cell carcinoma, head and neck squamous cell carcinoma, cervical cancer, small cell lung carcinoma, non-small cell lung carcinoma, mesothelioma, kidney cancer, liver cancer, brain cancer, skin cancer, melanoma, bladder cancer, and hematopoietic/blood cancer. These cancers can be from a human, other mammal, or a xenograft of human cancer cells removed from a non-human mammal (e.g., a mouse).

In some aspects, the tissue sample is a portion of a solid tumor or a complete tumor. Such a tissue sample containing tumor cells for use in the present invention may be obtained by any method as is known in the art, for example, by taking a biopsy from a patient. Suitable biopsies that may be employed in the present invention include, but are not limited to, blood draws into various tube types to collect blood-based tumors or circulating tumor cells, incisional biopsies, core biopsies, punch biopsies and fine needle aspiration (FNA) biopsies, as well as excisional biopsies.

Fine Needle Aspiration (FNA) biopsy is performed with a fine needle sometimes attached to a syringe and other times used independently. Aspiration biopsy or FNA may be employed in the present invention to obtain a cancer sample. FNA biopsy may be a percutaneous (through the skin) biopsy or alternatively through the lumen of an organ such as the bronchus, esophagus, stomach, or intestine. FNA biopsy is typically accomplished with a fine gauge needle (21 gauge or finer, e.g., 22 gauge or 25 gauge). The area is first cleansed and then usually numbed with a local anesthetic. The needle is placed into the region of organ or tissue of interest. Once the needle is placed a vacuum may be created with the syringe, or alternatively capillary action within the needle alone may be utilized, and multiple in and out needle motions are performed. The cells to be sampled are brought into the lumen of the needle and sometimes the hub of the needle through a micro-coring action of the bevel of the needle as it passes through the tissue. Three to six separate samples are usually made. Metastatic cancer sites such as lymph nodes and liver are good candidates for FNA biopsies. FNA procedures are typically done using ultrasound or computed tomography (CT) imaging.

A core needle biopsy (or core biopsy) is performed by inserting a small hollow needle through the skin and into the organ. The needle is then advanced within the cell layers to remove a sample or core. The needle may be designed with a cutting tip to help remove the sample of tissue. Core biopsy is often performed with the use of a spring-loaded gun to help remove the tissue sample. Core biopsy is typically performed under image guidance such as CT imaging, ultrasound or mammography. The needle is either placed by hand or with the assistance of a sampling device. Multiple insertions are often made to obtain sufficient tissue, and multiple samples are taken. Core biopsy is sometimes suction assisted with a vacuum device (vacuum-assisted biopsy). This method enables the removal of multiple samples with only one needle insertion. Unlike core biopsy, the vacuum-assisted biopsy probe is inserted just once into the tissue through a tiny skin nick. Multiple samples are then taken using a rotation of the sampling needle aperture (opening) and with the assistance of suction. Thus, core needle biopsy or vacuum-assisted needle biopsy may be employed in the present invention to obtain a tissue sample.

Endoscopic biopsy is a common type of biopsy that may be employed in the present invention to obtain a sample. Endoscopic biopsy is done through an endoscope (a fiber optic cable for viewing inside the body) which is inserted into the body along with sampling instruments. The endoscope allows for direct visualization of an area on the lining of the organ of interest; and collection or pinching off of tiny bits of tissue with forceps attached to a long cable that runs inside the endoscope of the sample. Endoscopic biopsy may be performed on, for example, the gastrointestinal tract (alimentary tract endoscopy), urinary bladder (cystoscopy), abdominal cavity (laparoscopy), joint cavity (arthroscopy), mid-portion of the chest (mediastinoscopy), or trachea and bronchial system (laryngoscopy and bronchoscopy), either through a natural body orifice or a small surgical incision. Endoscopic ultrasound-guided fine needle aspiration biopsy may also be performed on lung or mediastinal lymph nodes, pancreas, or liver using a trans-esophageal, trans-gastric or trans-duodenal approach.

Surface biopsy may be employed in the present invention to obtain a cancer sample. This technique involves sampling or scraping of the surface of a tissue or organ to remove cells. Surface biopsy is often performed to remove a small piece of skin.

In one aspect, the sample is from a subject with cancer.

The term “subject” as used herein refers to any individual or patient to which the subject methods are performed. Generally the subject is human, although as will be appreciated by those in the art, the subject may be an animal. Thus other animals, including vertebrate such as rodents (including mice, rats, hamsters and guinea pigs), cats, dogs, rabbits, farm animals including cows, horses, goats, sheep, pigs, chickens, etc., and primates (including monkeys, chimpanzees, orangutans and gorillas) are included within the definition of subject.

The term “treatment” is used interchangeably herein with the term “therapeutic method” or “therapy” and refers to both 1) therapeutic treatments or measures that cure, slow down, lessen symptoms of, and/or halt progression of a diagnosed pathologic conditions or disorder, and 2) and prophylactic/preventative measures. Those in need of treatment may include individuals already having a particular medical disorder as well as those who may ultimately acquire the disorder (i.e., those needing preventive measures).

In some aspects administration can be in combination with one or more additional therapeutic agents. The phrases “combination therapy”, “combined with” and the like refer to the use of more than one medication or treatment simultaneously to increase the response.

In various aspects, the cancer is selected from the group consisting of colorectal, esophageal, stomach, lung, mesothelioma, prostate, uterine, breast, skin, endocrine, melanoma, urinary, pancreas, ovarian, cervical, head and neck, liver, bone, biliary tract, small intestine, hematopoietic/blood cancers (myeloma, leukemia, and lymphoma), vaginal, testicular, anal, kidney, brain, eye cancer, leukemia, lymphoma, soft tissue, melanoma, mixed types, primary of unknown origin, and metastases thereof. In other aspects, the sample is from single or multiple tumor tissue of any type of tumor. In many aspects, the tumor sample is from a solid tumor.

In another aspect, the tumor sample is obtained by fine needle aspiration, core biopsy, collecting circulating tumor cells, surgical excision, or other tumor sample acquisition method.

In other aspects, the method further includes stratification of patients on their predicted responsiveness to a therapeutic agent or therapeutic regimen band providing a positive or negative treatment value which corresponds to positive or negative clinical outcome, respectively.

In some aspects, the method is further used to identify, monitor, predict the existence of and characterize functional sub-populations (heterogeneity) of cancer cells, which can be used to predict a positive or negative response to ICI monotherapies and/or combination therapies; the identification and characterization of functional sub-populations can be used to predict and identify the mechanisms of innate and acquired resistance to ICI monotherapies and/or combination therapies. This information can be used to inform the creation and tailoring of ICI monotherapies and/or combination therapies regimens to the individual patient.

In one aspect, the biological sample is a cancer cell or a cancer cell subpopulation, including but not limited to cancer stem cells, wherein the cancer stem cells express one or more of CD133, CD44, ABCG2, ALDH1A1 and/or do not stain with Hoechst.

As used herein, the term “cancer stem cell” refers to cells found within tumors or hematological cancers that possess characteristics associated with normal stem cells, specifically the ability to give rise to all cell types found in a particular cancer sample, and the self-renewal properties. Therefore, such cells are hypothesized to persist in tumors as a distinct population and cause relapse and metastasis by giving rise to new tumors; and the development of specific therapies targeting cancer stem cells holds hope for improvement of survival and quality of life of cancer patients, especially for patients with metastatic disease. Several biomarkers have been identified as specific cancer stem cell biomarkers. Examples of biomarkers of cancer stem cells include CD133, CD44, ABCG2, and/or ALDH1A1.

In another aspect, the biological sample is immune cells or immune cell subpopulations, including but not limited to T cells, CD8+ T cells, exhausted T cells, active T cells, B cells, NK cells, dendritic cells, myeloid derived suppressor cells, macrophages, granulocytes, and mast cells. In yet another aspect, the modulator is a biologic, a biosimilar, a derivative thereof, a mutant thereof, a peptide thereof, a fragment thereof, an analog thereof or a mimetic thereof; and the derivative, mutant, peptide, fragment, analog or mimetic of the modulator enhances or decreases half-life and/or target binding, and has a similar effect as the intact modulator.

In other aspects, the modulator is an interferon. In various aspects, the interferon is IFN-α, IFN-β, or IFN-γ.

In another aspect, the modulator is an Interleukin. In various aspects, the interleukin is IL-1, IL-2, IL-4, IL-6, IL-7, IL-8, IL-10, IL-12, IL-15, IL-17, IL-18, IL-21, IL-22, IL-23, IL-27, or IL-35.

In other aspects, the modulator is FGF, EGF, VEGF, prostaglandins (e.g. prostaglandin (PG) E2 (PGE2), prostacyclin (PGI2), prostaglandin D2 (PGD2) and prostaglandin F2α (PGF2α)), IDO, GM-CSF, TNF-alpha, TGF-beta, a derivative thereof, a mutant thereof, a peptide thereof, a fragment thereof, an analog thereof or a mimetic thereof; and the derivative, mutant, peptide, fragment, analog or mimetic of the modulator enhances or decreases half-life and/or target binding, and has a similar effect as the intact modulator.

In one aspect, the biological sample/cancer subject is treated in a manner to alter conditions such as abundance of proteins, carbohydrates, and lipids, and molecules found within fetal bovine serum, growth factors, hypoxia, oxidative stress, and/or physical exercise/stress, or the sample is modulated using a patients' “milieu” from a tumor biopsy optionally consisting of various cell types such as fibroblasts, inflammatory and immune cells, and soluble factors derived from tumor and associated cells; in yet other aspects, the sample is modulated using the patients' own immune cells such as those derived from tumors (e.g. T cells, B cells, NK (Natural Killer) cells, macrophages and dendritic cells, myeloid derived suppressor cells and granulocytes) or peripheral blood immune cells (e.g. T cell, B cells, NK cells, monocytes, dendritic cells, tumor cells, granulocytes, NK cells).

As used herein “fetal bovine serum” or “FBS” refers to serum collected from the blood drawn of a bovine fetus via a closed system of collection. FBS is the most widely used serum-supplement for the in vitro cell culture of eukaryotic cells, mainly because it has a very low level of antibodies and contains more growth factors, allowing for versatility in many different cell culture applications. The globular protein, bovine serum albumin (BSA), is a major component of fetal bovine serum amongst the rich variety of proteins present in fetal bovine serum. As FBS is not a fully defined media component, and as such may vary in composition between batches.

In some aspects, multiple modulators are added together, sequentially or alternately.

In one aspect, the modulator is a reactive oxygen species or a free radical molecule, radiation therapy such as x-rays, gamma rays, or charged particles.

As used herein, “reactive oxygen species” or “ROS” refers to chemically reactive chemical species containing oxygen. ROS are formed as a natural byproduct of the normal metabolism of oxygen and have important roles in cell signaling and homeostasis. However, during times of environmental stress or oxidative stress (e.g., UV or heat exposure), ROS levels can increase dramatically. This may result in significant damage to cell structures. Examples of ROS include peroxides, superoxide, hydroxyl radical, singlet oxygen, and alpha-oxygen.

In one aspect, the modulator is a phenothiazine such as prochlorperazine.

As used herein, “phenothiazine” refers to an antipsychotic drug. Antipsychotics can be used to reduce hallucinations and delusions associated with psychosis. Some phenothiazines (such as prochlorperazine and chlorpromazine) are also effective at relieving other symptoms unrelated to psychosis, such as nausea, vomiting, prolonged hiccups, tetanus symptoms and hyper-excitable behavior in children. Non-limiting examples of phenothiazine include: prochlorperazine, chlorpromazine, trifluoperazine, fluphenazine, thioridazine, perphenazine and mesoridazine.

In other aspects, the modulator is a chemotherapeutic agent. The term “chemotherapeutic agent” as used herein refers to any therapeutic agent used to treat cancer. In various aspects, the chemotherapeutic agent is an alkylating agent, such as a mustard gas derivative, including but not limited to mechlorethamine, cyclophosphamide, chlorambucil, melphalan, and ifosfamide; an ethylenimine, including but not limited to thiotepa and hexamethylmelamine; an alkyl sulfonate, including but not limited to busulfan; a hydrazine and/or triazine, including but not limited to altretamine, procarbazine, dacarbazine and temozolomide; or a nitrosureas, including but not limited to carmustine, lomustine and streptozocin; a metal salt, including but not limited to carboplatin, cisplatin, and oxaliplatin.

In some aspects, the chemotherapeutic agent is a plant alkaloid, such as a vinca alkaloid, including but not limited to vincristine, vinblastine and vinorelbine; a taxane, including but not limited to paclitaxel and docetaxel; a podophyllotoxin, including but not limited to etoposide and tenisopide; or a camptothecin analog, including but not limited to irinotecan and topotecan.

In other aspects, the chemotherapeutic agent is an antitumor antibiotic, such as an anthracycline, including but not limited to doxorubicin, daunorubicin, epirubicin, mitoxantrone, and idarubicin; a chromomycin, including but not limited to dactinomycin and plicamycin; mitomycin or bleomycin.

In other aspects, the chemotherapeutic agent is an antimetabolite, such as a folic acid antagonist, including but not limited to methotrexate and pemetrexed; a pyrimidine antagonist, including but not limited to 5-fluorouracil, floxuridine, cytarabine, capecitabine, and gemcitabine; or a purine antagonist, including but not limited to 6-mercaptopurine and 6-thioguanine an adenosine deaminase inhibitor, including but not limited to cladribine, fludarabine, nelarabine and pentostatin.

In yet another aspect, the chemotherapeutic agent is a topoisomerase inhibitor, such as a topoisomerase I inhibitors including but not limited to irinotecan and topotecan; or a topoisomerase II inhibitors including but not limited to amsacrine, etoposide, etoposide phosphate, and teniposide.

In various aspects, the modulator is a molecularly targeted agent, such as an inhibitor of EGFR, including but not limited to erlotinib, cetuximab, osimertinib, vandetanib, panitumumab, necitumumab, gefitinib and afatinib; an inhibitor of ALK, including but not limited to alectinib, brigatinib, ceritinib, and crizotinib; an inhibitor of one or more members of the VEGF family (VEGF ligand, VEGFR, VEGFR2, VEGFA/B, VEGFR1/2/3), including but not limited to bevacizumab, pazopanib, ramucirumab; sorafenib, Ziv-aflibercept, lenvatinib, axitinib, vandetanib, cabozantinib, and regorafenib; an inhibitor of KIT, including but not limited to axitinib, cabozantinib, imatinib, pazopanib, regorafenib; an inhibitor of HER2, including but not limited to lapatinib, neratinib, pertuzumab, dacomitinib, trastuzumab and ado-trastuzumab emtansine; an inhibitor of CDK4 and CDK6, including but not limited to palbociclib and ribociclib; an inhibitor of BRAF, including but not limited to dabrafenib and vemurafenib; an inhibitor of PARP, including but not limited to niraparib, olaparib, and rucaparib; an inhibitor of one or more members of the JAK family, including but not limited to ruxolitinib and tofacitinib; an inhibitor of mTOR, including but not limited to everolimus and temsirolimus; an inhibitor of MEK, including but not limited to cobimetinib and trametinib; an inhibitor of ERK, including but not limited to ulixertinib; an inhibitor of PDGFR (or part thereof including PDGFRα and PDGFRβ), including but not limited to axitinib, imatinib, olaratumab, pazopanib, regorafenib, and sorafenib; an inhibitor of RAF, including but not limited to regorafenib and sorafenib; an inhibitor of RET, including but not limited to regorafenib, vandetanib and cabozantinib; an inhibitor of MET, including but not limited to cabozantinib and crizotinib; an inhibitor of ROS1, including but not limited to crizotinib; or an inhibitor of any one of the following targets: PIGF, PTCH, Smoothened, RANKL, and B4GALNT1, (e.g. Ziv-aflibercept, vismodegib, sonidegib, denosumab, dinutuximab); an inhibitor of the STAT family, including but not limited to danvatirsen, AZD9150 and TTI-101; an inhibitor of HDAC family (including HDAC3 and HDAC6), including but not limited to nexturastat A, ricolinostat, trichostatin A, vorinostat, panobinostat, vaproic acid, belinostat, and entinostat; an inhibitor of BET family (including BRD1 and BRD4), including but not limited to RG6146, ABBV-075, OTX015/MK-8628, GSK2820151/I-BET151, CC-90010, PLX51107, and LY294002; an inhibitor of NTRK, including but not limited to larotrectinib and entrectinib; an inhibitor of Bcl-2, including but not limited to venetoclax, navitoclax, and obatoclax; an inhibitor of ATM, including but not limited to AZD1390, AZD0156, and M3541; an inhibitor of ATR, including but not limited to AZD6738 and M6620; an inhibitor of A2AR, including but not limited to AZD4635 and CPI-444; an inhibitor of WEE1, including but not limited to adavosertib and ADZ1775; an inhibitor of FGFR, including but not limited to erdafitinib, pemigatinib and TAS-120; or an autophagy influencer.

As used herein “autophagy” refers to the physiological regulated cellular mechanism that removes unnecessary or dysfunctional components and allows the orderly degradation and recycling of cellular components. The term autophagy may refer to any of the three forms of autophagy macroautophagy, microautophagy, and chaperone-mediated autophagy (CMA) without any specific distinction. “Autophagy influencers” are meant to refer to any molecule, agent or drug that can affect autophagy, whether it enhances or inhibits autophagy. In some aspects, the autophagy influencer is metformin, melatonin, trehalose, spermidine, spermine, azithromycin, chloroquine, or chloramphenicol.

In one aspect, the biomarker is measured on tumor cells, stromal cells (e.g. fibroblasts) or immune cells (T cells, B cells, NK cells, dendritic cells, myeloid derived suppressor cells, macrophages, granulocytes, and mast cells), or subpopulation thereof. In various aspects, the subpopulation includes cancer stem cells that express one or more of CD133, CD44, ABCG2, and/or ALDH1A1. In other aspects, the biomarker is the localization, and/or level, and/or state of a molecule or molecules. In various aspects, the molecule(s) being measured is a protein or a nucleic acid; the state of the molecule(s) being measured is phosphorylation, acylation, alkylation, amidation, glypiation, glycation, glycosylation, ubiquitination, degradation product(s), truncation, mutation status, binding of the molecule(s) to promoters that induce PD-1 ligand expression including GAS (Gamma Activated Sequence) promoter/enhancer regions and the promoter of PD-1 ligand genes, methylation status, chromatin modification of the promoter/enhancer region or the status of the SWI/SNF complexes; the localization of the molecule(s) being measured is extracellular or cellular, wherein cellular localization includes intracellular, compartmentalized (e.g. Golgi, endoplasmic reticulum, lysosomal, endosomal, exosomal, mitochondrial, vacuole, cytoslic), nuclear or nucleoli, or membrane (e.g. plasma, nuclear or other organelle membrane) bound including measurements of endocytosis and exocytosis.

In many aspects, the molecule(s) being measured is PD-1 or a PD-1 ligand (PD-L1 or PD-L2), AZAR, CD40, CD40L, galectin-9, IDO1, CTLA-4, KIRs, 4-1BB, GITR, T cell immunoglobulin mucin 3 (TIM-3), LAG-3 (CD223), Killer immunoglobulin-like receptors (KIRs), VISTA, Adenosine, MHC I and/or MHC II; the molecule(s) being measured is in a pathway that influences PD-1 ligand expression including the immediate target or downstream target of an Interleukin; is FGF, EGF, VEGF, prostaglandins (e.g. prostaglandin (PG) E2 (PGE2), prostacyclin (PGI2), prostaglandin D2 (PGD2) and prostaglandin F2α (PGF2α)), IDO, or GM-CSF; is in TNF-alpha, TGF-beta, the IFN alpha, beta, and gamma response pathways; in the JAK/STAT pathway, the EGFR response pathway, the PI3K pathway, the MAPK pathway, and/or the mevalonate pathway.

As used herein, the mevalonate pathway refers to the biosynthetic pathway that produces terpenes and steroids, having mevalonic acid (MVA or dihydroxymethylvalerolactone) as a precursor. The carboxylate anion of mevalonic acid, which is the predominant form in biological environments, is known as mevalonate. Drugs like statins (which lower levels of cholesterol) stop the production of mevalonate by inhibiting HMG-CoA reductase.

In various aspects, the molecule(s) being measured is IFN-alpha, beta and/or gamma receptors; is from the STAT family (Signal Transducer and Activator of Transcription) such as STAT-1, STAT-2, STAT-3 or STAT-4; is from the JAK family (Janus Kinase), such as JAK-1 or JAK-2; is from the IRF family (Interferon Regulatory Factors), such as IRF-1 or IRF-9; is EGFR; is from the PI3K pathway; is PTEN, PI3K, PDK1, AKT, mTOR, S6, S6-Kinase, CREB, GSK3B, mTORC1 or mTORC2; is from the MAPK pathway, such as Src, Fak, Ras, Raf, Mek, Erk, CREB, Sos-1, SHC, NFkB, cMyc, ELK-1, Tristetraprolin (TTP), c-Fos or c-Jun; is a receptor tyrosine kinase (e.g. RET, EGFR, MET, KIT, ALK, HER2, ROS1, JAK2, VEGFR, or PDGFR); is Hif-1a or Hif-2a; is an interleukin receptor (GP130, IL1R, TNFR, IL2Ra/b/g); or is a direct or indirect target of interferon, interleukin or TNF receptors (TRAF2, TRAF-5, TRAF-6, I-Kappa B, MyD88, IRAK1, IRAK2, IRAK3, IRAK4,MKK, 1 KB, IKK, NFATc1, p38, p48, JNK, AP1, IRS, SHC, GRB2, SOS); part of the mevalonate pathway (ARF6 and/or AMAP1).

In other aspects, the biomarkers are markers of autophagy, selected from the group consisting of LC3I, LC3II, LC3/LC3II/LC3II ratio, mTOR, AMPK, p62/SQSTM1, ATM, UNC51-like kinase-1, -2, and -3.

In many aspects, the biomarker being measured is the abundance of endosomes or exosomes, the contents of endosomes or exosomes (e.g. an Interleukin, FGF, EGF, VEGF, prostaglandins (e.g. prostaglandin (PG) E2 (PGE2), prostacyclin (PGI2), prostaglandin D2 (PGD2) and prostaglandin F2α (PGF2α)), IDO, GM-CSF, TNF-alpha, and TGF-beta) or the abundance and release of an Interleukin, FGF, EGF, VEGF, prostaglandins (e.g. prostaglandin (PG) E2 (PGE2), prostacyclin (PGI2), prostaglandin D2 (PGD2) and prostaglandin F2α (PGF2α)), IDO, GM-CSF, TNF-alpha, and TGF-beta.

In one aspect, the ICI response biomarkers are combined with p53 mutation status, tumor mutation burden.

As used herein, the term “p53 status” refers to mutational status of a subject regarding the gene TP53, encoding the protein p53. p53 is described as “the guardian of the genome” because of its role in conserving genome stability by preventing genome mutation; it is thus crucial in multicellular organisms, where it prevents cancer formation. Therefore, p53 functions as a tumor suppressor, and the mutational status of p53 has crucial implication in the management and care of cancer patients.

In some aspects, the ICI response biomarkers are combined with the abundance, phenotypic and functional activity (cell proliferation, survival, and cytotoxicity) of tumor-infiltrating immune cells (e.g. myeloid dendritic cells, macrophages, granulocytes, mast cells and tumor-infiltrating lymphocytes (TILs); T helper cells, T cytotoxic cells, T regulatory cells, B cells and NK cells); the ICI response biomarkers are combined with PD-1 baseline level to predict a positive response to anti-PD-1 therapy for patients with PD-L1 negative tumors; or the ICI response biomarkers are combined with markers of immune senescence (diminished cell proliferation, cytokine secretion (such as IFNγ, TNFα and IL-2), display of PD-1, Tim3 and LAG3), and lysosomal-associated membrane protein-1 (also referred to as CD107a) indicative of T cell impairment and immune escape.

In other aspects, the response biomarkers are combined with static levels of the biomarkers including PD-1, PD-1 ligand, AZAR, CD40, CD40L, galectin-9, IDO1, CTLA-4, KIRs, 4-1BB, GITR, T cell immunoglobulin mucin 3 (TIM-3), LAG-3 (CD223), Killer immunoglobulin-like receptors (KIRs), VISTA, Adenosine, MHC I and MHC II. In yet another aspect, the ICI response biomarkers are combined with the size of the MHC-bound peptides.

In one aspect, the molecules being measured are an Interleukin, FGF, EGF, VEGF, prostaglandins (e.g. prostaglandin (PG) E2 (PGE2), prostacyclin (PGI2), prostaglandin D2 (PGD2) and prostaglandin Fat (PGF2α)), IDO, GM-CSF, TNF-alpha, and TGF-beta, which are generated by the tumor cells or cells found in the tumor microenvironment.

In many aspects, the biomarker being measured is Toll-like receptor (TLR), NKG2D, B3NT3, or NFkBP65; SWI/SNF complex, BRG1, BAF180, CTCF, an miRNA, such as miRNA-570, miRNA-513 or miRNA-155.

In other aspects; the response biomarkers are combined with the patient race, gender, age or age-related biomarkers, such as follicle stimulating hormone, biopsy type, tumor stage, tumor type and/or histological categorization (e.g. cell cycle status, cell type and differentiation status) of the sample.

In some aspects, the biomarker(s) are measured using immunoassays, multiplexed assays, PCR, transcription factor assays, nucleic acid or sequencing/mutation testing. In one aspect, the assay is an immunoassay (e.g. western blot, dot blot, ELISA, immunohistochemistry, immunocytochemistry, immunofluorescence).

In various aspects, the expression level of a biomarker is normalized against a normalization biomarker selected from the group consisting of actin, GAPDH, vinculin, tubulin, Na/K-ATPase, HSP90 and a histone.

As used herein, the term “normalization biomarker” refer to any biomarker that can be used as a reference in a given assay, in order to allow proper quantification or the biomarker of interest, by comparing said expression levels of a biomarker of interest to the expression level of a normalization marker. Normalization biomarkers, or reference biomarkers are well known in the art, and various reference biomarkers are available; the most crucial aspect being that the reference biomarker must be stable. Non-limiting examples of normalization biomarkers include actin, GAPDH, vinculin, tubulin, Na/K-ATPase, HSP90, histone, ubiquitin and 18S.

In other aspects, the analytical technique is a multiplexed assays (e.g. flow cytometry, microarrays, and bead-based (e.g. Luminex multiplex assays)); the analytical techniques include all forms of PCR, including but not limited to qPCR, RT-PCR, real-time PCR and endpoint PCR; the analytical technique is a transcription factor identification assays (e.g. protein arrays, chromatin immunoprecipitation (CHIP) and CHIP-seq assays, DNA precipitation and DIP-seq assays, microsphere assays, DNAse sensitivity and gel shift assays; and DNA hypersensitivity assay); and the analytical techniques include all forms of sequencing DNA and RNA molecules, whole genome, exome, or specific genes only, including but not limited to massively parallel signature sequencing (MPSS), 454 pyrosequencing, Illumina (Solexa) sequencing, SOLiD sequencing, Ion Torrent semiconductor sequencing, Heliscope single molecule sequencing, single molecule real-time (SMRT) sequencing, sequencing by hybridization, and sequencing with mass spectrometry.

In yet another aspect, the biological sample is processed in part, or entirely, using one or more manual methods and/or automated systems.

In some aspects, a biomarker or panel of biomarkers selected from PD-1, PD-L1, PD-L2, AZAR, CD40, CD40L, galectin-9, IDOL CTLA-4, KIRs, 4-1BB, GITR, TIM-3, LAG-3, lysosomal-associated membrane protein-1, KIRs, VISTA, Adenosine, MHC I, MHC II, STAT-1, STAT-2, STAT-3, STAT-4, JAK-1, JAK-2, IRF-1, IRF-9, EGFR, PTEN, PI3K, PDK1, AKT, mTOR, S6, S6-Kinase, CREB, GSK3B, mTORC1, mTORC2, Src, Fak, Ras, Raf, Mek, Erk, CREB, Sos-1, SHC, NFkB, cMyc, ELK-1, Tristetraprolin, c-Fos, c-Jun, RET, EGFR, MET, KIT, ALK, HER2, ROS1, JAK2, VEGFR, PDGFR, Hif-1a, Hif-2a, GP130, IL1R, TNFR, IL2Ra/b/g, TRAF2, TRAF-5, TRAF-6, I-Kappa B, MyD88, IRAK1, IRAK2, IRAK3, IRAK4, MKK, 1 KB, IKK, NFATc1, p38, p48, JNK, AP1, IRS, SHC, GRB2, SOS, Toll-like receptor (TLR), NKG2D, B3NT3, NFkBP65, SWI/SNF complex, BRG1, BAF180, CTCF, miRNA-570, miRNA-513, miRNA-155, IFN-α, IFN-β, or IFN-γ, IL-1, IL-2, IL-4, IL-6, IL-7, IL-8, IL-10, IL-12, IL-15, IL-17, IL-18, IL-21, IL-22, IL-23, IL-27, IL-35, FGF, EGF, VEGF, prostaglandin E2, prostacyclin, prostaglandin D2, prostaglandin Fat, IDO, GM-CSF, TNF-alpha, TGF-beta, tumor mutation burden, p53 mutation status, the abundance, phenotypic and functional activity, including cell proliferation, survival, and cytotoxicity, of tumor-infiltrating immune cells selected from the group consisting of myeloid dendritic cells, macrophages, granulocytes, mast cells and tumor-infiltrating lymphocytes (TILs); T helper cells, T cytotoxic cells, T regulatory cells, B cells and NK cells, patient race, gender and age or age-related biomarkers such as follicle-stimulating hormone, biopsy type, tumor stage, tumor type and/or histological categorization including cell cycle status, cell type and differentiation status of the sample, actin, GAPDH, vinculin, tubulin, Na/K-ATPase, HSP90 or a histone, is used to predict the likelihood of response including of detecting the expression levels of biomarkers in a specimen as compared with a non-responsive group and a responsive group of samples; and the expression levels of the biomarkers are evaluated by applying a statistical method such as receiver operating characteristic (ROC) curve cut value analysis, regression analysis, discriminant analysis, classification tree analysis, random forests, support vector machine, OneR, kNN and heuristic naive Bayes analysis, neural nets and variants thereof.

In other aspects, the predicting of likelihood of response is performed by a software classification algorithm.

In one aspect, the modulator includes one or more therapeutic PD-1 or PD-L1 antibodies selected from pembrolizumab, nivolumab, cemiplimab, atezolizumab, avelumab, or durvalumab.

In another aspect, the biomarker includes the ability of the therapeutic antibodies to detect PD-1 or PD-L1 or interfere with the interaction of PD-1 and PD-L1.

As used herein, “therapeutic PD-1 or PD-L1 antibodies” refers to antibodies that are approved, or clinically tested for the treatment of patients. Therapeutic PD-1 or PD-L1 antibodies are antibodies that are recognized for their ability to block the interaction of PD-L1 with the PD-1 receptor, and to prevent the cancer cells from evading the immune system. Non-limiting examples of approved or clinical developed PD-1 or PD-L1 antibodies include: pembrolizumab, nivolumab, cemiplimab, atezolizumab, avelumab, durvalumab, spartalizumab, camrelizumab (SHR1210), sintilimab, tislelizumab, toripalimab, dostarlimab, INCMGA00012, AMP-224, AMP-514, KN035 and CK-301.

By “one or more”, it is meant that the modulator or biomarker can include several different antibodies to be used at a same time. For example, the modulator can include 1, 2, 3, 4 or more therapeutic PD-1 or PD-L1 antibodies or the biomarker can include comparison of detection of PD-1 and PD-L1 by 1, 2, 3, 4 or more therapeutic PD-1 or PD-L1 antibodies.

In various aspects, the therapeutic antibody is conjugated to a single detection molecule selected from fluorescein isothiocyanate (FITC), Qdot 655, horseradish peroxidase (HRP), or alkaline phosphatase (AP).

In some aspects, multiple therapeutic PD-1 or PD-L1 antibodies are used as a modulator on a single sample. For example, 1, 2, 3, 4 or more PD-1 or PD-L1 antibodies can be used as a modulator on a single sample.

In one aspect, each therapeutic antibody is conjugated to a unique detection molecule selected from FITC, tetramethylrhodamine (TRITC), Qdot 655, HRP, AP, or 3,3′-Diaminobenzidine (DAB). For example, a first PD-1 antibody can be conjugated to FITC, and a second PD-1 antibody can be conjugated to TRITC, a third PC-1 antibody can be conjugated to Odot 655, a first PD-L1 antibody can be conjugated to HRP, a second PD-L1 antibody can be conjugated to AP, and a third PD-L1 antibody can be conjugated to DAB.

In some aspects, the biomarker is the ability for PD-1 or PD-L1 protein to bind to PD-L1 or PD-1, respectively.

In other aspects, a PD-1 or PD-L1 protein is conjugated to a single detection molecule selected from FITC, TRITC, Qdot 655, HRP, DAB, or AP.

In one aspect, the biomarker is the intensity of detection molecules in the sample.

Presented below are examples discussing the use of modulator to_alter the level, form, and localization of immune checkpoint proteins to produce a more robust response to ICI and/or to identify, monitor, prognose, and predict responsiveness to an ICI therapy contemplated for the discussed applications. The following examples are provided to further illustrate the embodiments of the present invention, but are not intended to limit the scope of the invention. While they are typical of those that might be used, other procedures, methodologies, or techniques known to those skilled in the art may alternatively be used

### EXAMPLES

### Example 1

### Materials and Methods

Cell Culture and Reagents: The cell lines H2170, H2126, A375 and HCC827 were obtained from the American Type Culture Collection (Manassas, Va.). All the lung cancer cell lines were maintained in RPMI-1640 GlutaMAX in the presence of 5% fetal bovine serum (FBS), 50 U/ml penicillin/streptomycin and 1 mM sodium pyruvate were maintained in a humidified incubator at 37° C. with 5% carbon dioxide.

Sub-cultivation and harvesting of all cell lines was performed with Acctuase (GIBCO, Life Technologies, Inc.) at 37 C for 5-10 min. Erlotinib hydrochloride was purchased from Selleck Chemicals. Recombinant human TNF-a was purchased from BD Biosciences (CA, USA).

Antibodies, PD-L1 were purchased from cell signaling (13684S, 15165S) band Abcam (ab28-8); phospho-tyrosine (cell signaling).

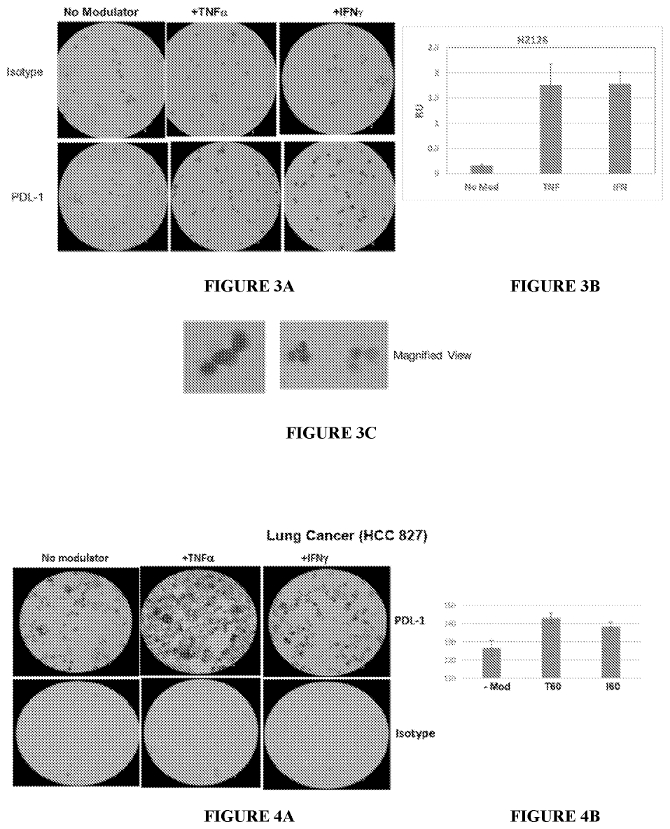

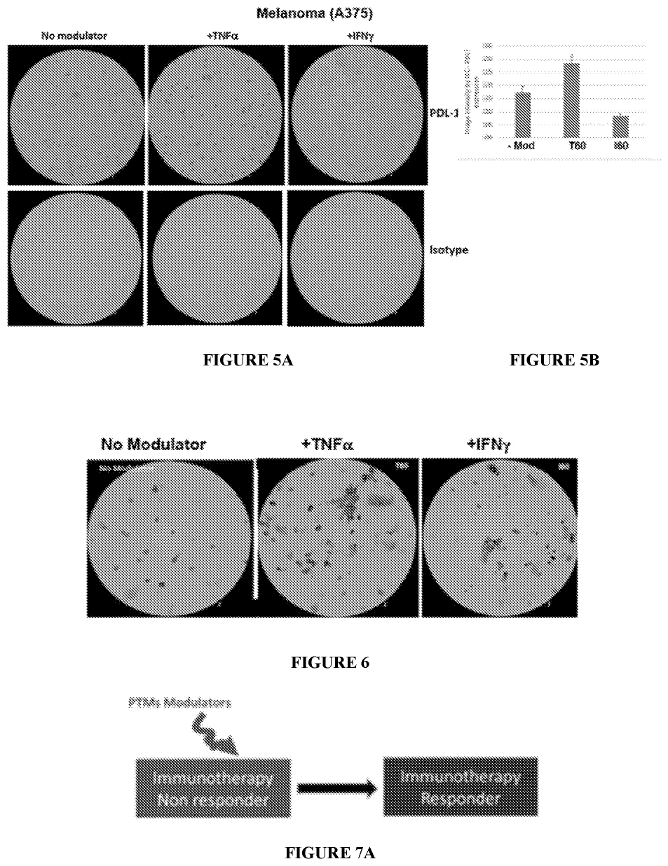

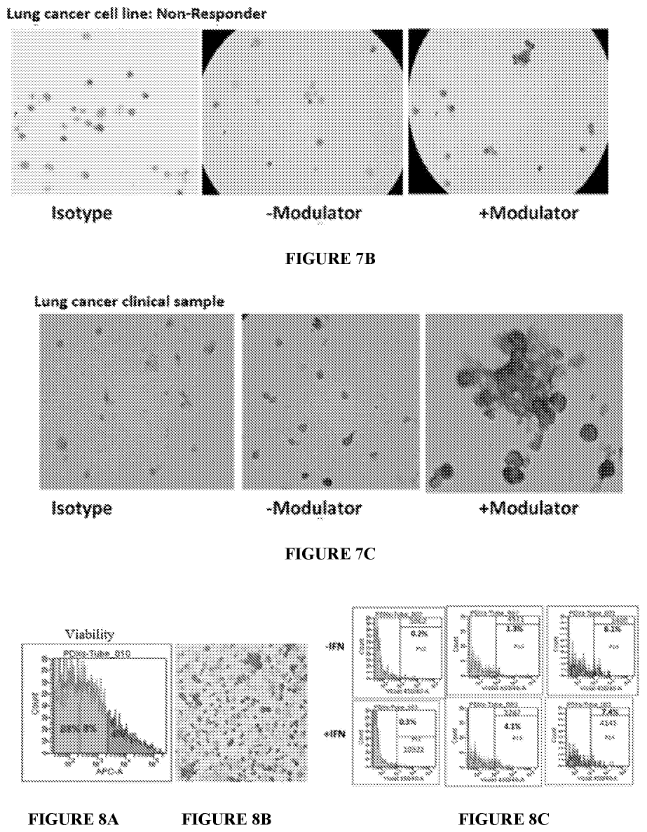

Immunocytochemistry (ICC) Staining: After the treatment with modulator(s) (IFN γ (100 ng/ml); TNF α (10 ng/ml) for specific timing in a humidified incubator at 37° C. with 5% carbon dioxide, the cells were pellet down and washed twice with PBS. The cells were spotted on poly-lysine coated slides and processed for ICC staining. Ambay kit (Catalog 410011A) purchased from Ambay Immune Sensors and Controls, LLC (AISC) (MD, USA) was used to perform ICC staining as per the kit instructions and Poly-HRP from Biogenix (CA, USA) was used to enable signal amplification and detection of specific bound antibody targets. A respective isotype antibody control was kept in parallel. Counterstaining was performed before mounting and analysis (FIGS. 2-7).

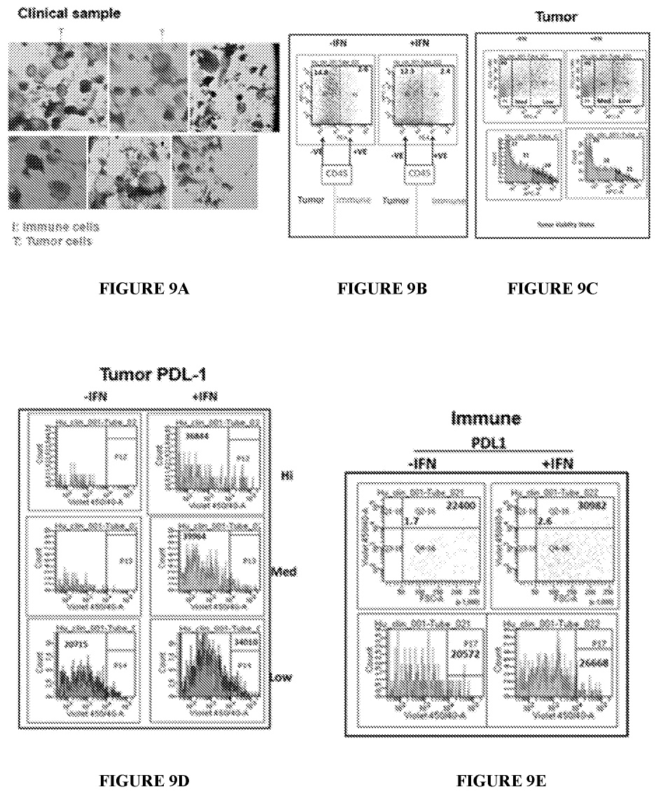

Flow Cytometry: Six-color flow cytometry was performed on BD FACS Aria II (BD Biosciences) using FITC, PE, PerCP, BV 420, BV 650 allophycocyanin-Cy7, and allophycocyanin as fluorochromes (FIGS. 8, 9, 14, and 15A). After the treatment with modulator(s) (IFN γ (100 ng/ml); TNF α (10 ng/ml) for specific timing in a humidified incubator at 37° C. with 5% carbon dioxide. The cells were pelleted down and washed twice with PBS before proceeding with the staining for flow cytometry. The cells were stained for different cell surface and intracellular markers as per the AISC kit instructions (Catalog #10011; purchased from AISC (MD, USA)). LIVE/DEAD LIVE/DEADTM Fixable Far Red Dead Cell Stain Kit (ThermoFisher cat #L34973) was used to distinguish live cells from dead cells. The Invitrogen ArC Amine Reactive Compensation Bead Kit (ThermoFisher cat #A10346) was used to demonstrate a consistent monitoring of viability status. Both the cells stained with single-color fluorochromes and beads (catalog #51-90-9001291, 51-90-9001229) were used for setting up compensation, background values were established with isotype controls, and data were analyzed using FACSDiva software (BD Biosciences).

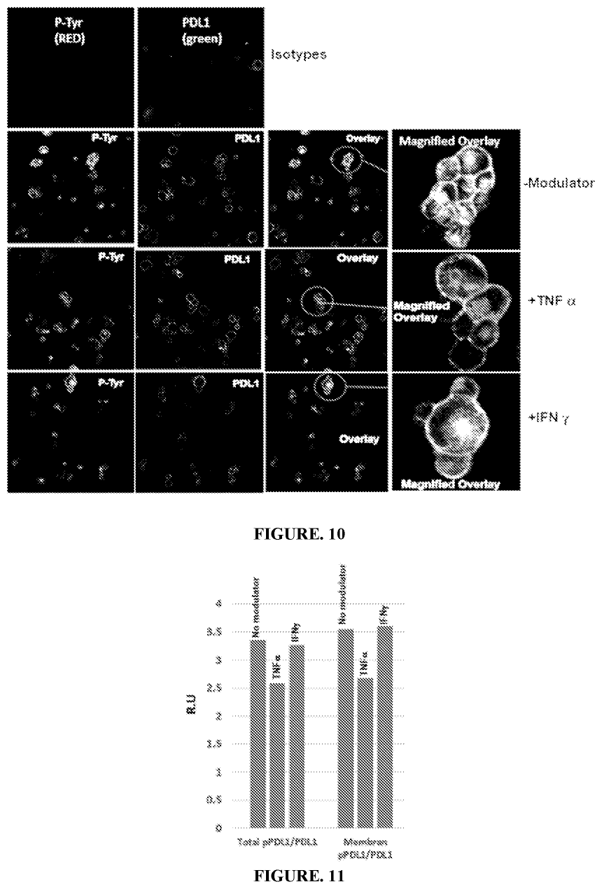

Confocal Microscopy: After the treatment with modulator(s) (IFN γ (100 ng/ml); TNF α (10 ng/ml) for specific timing in a humidified incubator at 37° C. with 5% carbon dioxide. The cells were pellet down and washed twice with PBS before proceeding with the staining for confocal microscopy (FIGS. 10 and 11). Cells were surface stained for PD-L1 and PTyr using AISC kit, according to manufacturer's instructions (Catalog #10011). Isotype control staining was performed in parallel. Cells were analyzed with a confocal microscope (Leica).

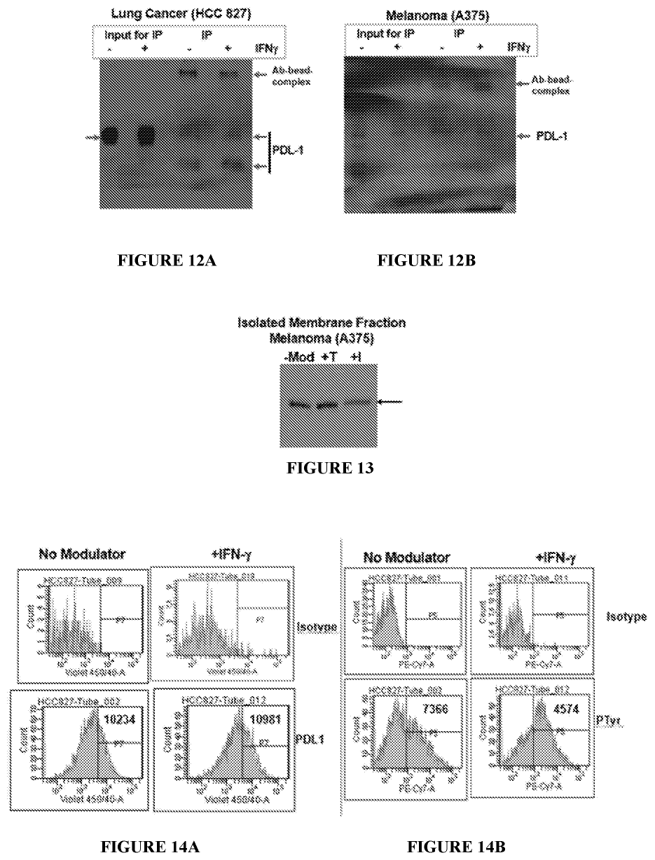

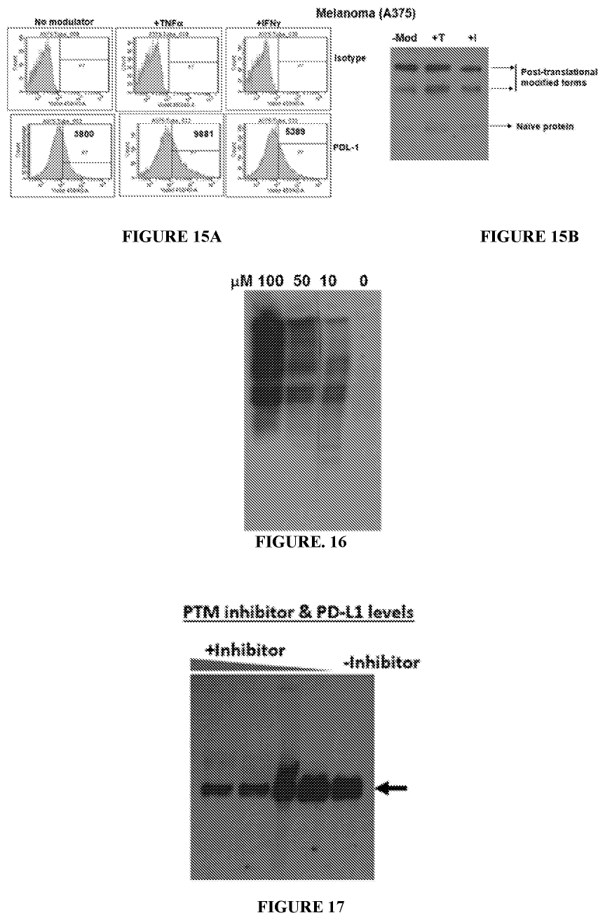

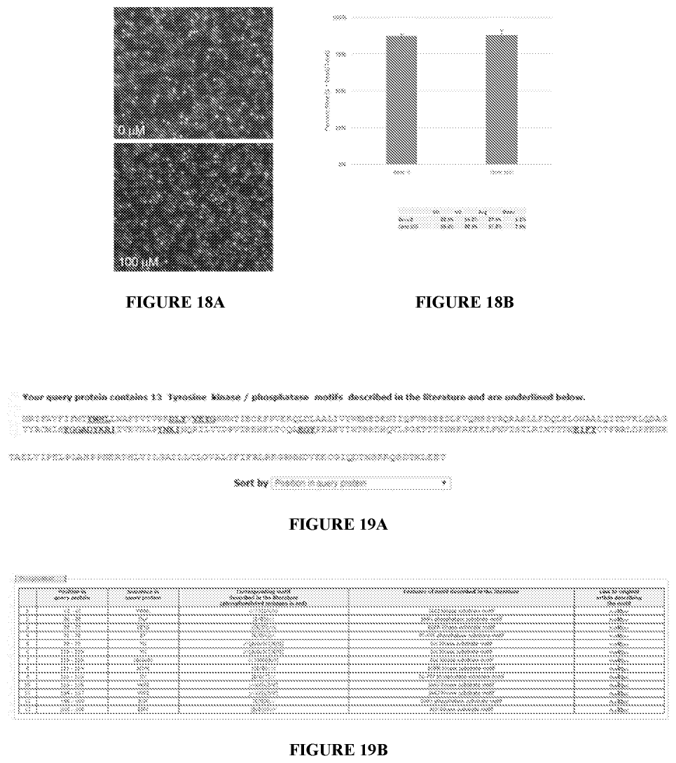

Immunoprecipitation Assay: After the treatment with modulator, IFN γ (100 ng/ml) for 60 min in a humidified incubator at 37° C. with 5% carbon dioxide, the cells were pelleted down, washed twice with PBS and lysed with ice-cold BlastR lysis buffer containing a cocktail of NEM, TSA, Na3VO4, and protease inhibitors (Cytoskeleton, CO). Samples were immunoprecipitated using pY PTM identification, Signal-Seeker kit, according to the manufacturer's protocol (Cytoskeleton, CO). The appropriate amount of pY (APY03) was added to the respective samples for 1 to 2 hours at 4° C. on shaker. After incubation, the affinity beads from each sample were pelleted and washed 3× with BlastR wash buffer. Bound proteins were eluted using the elution buffer and spin columns in the Signal-Seeker kits, and PTM modified target proteins were detected by western immunoblotting (FIGS. 12, 15B,16 and 17).

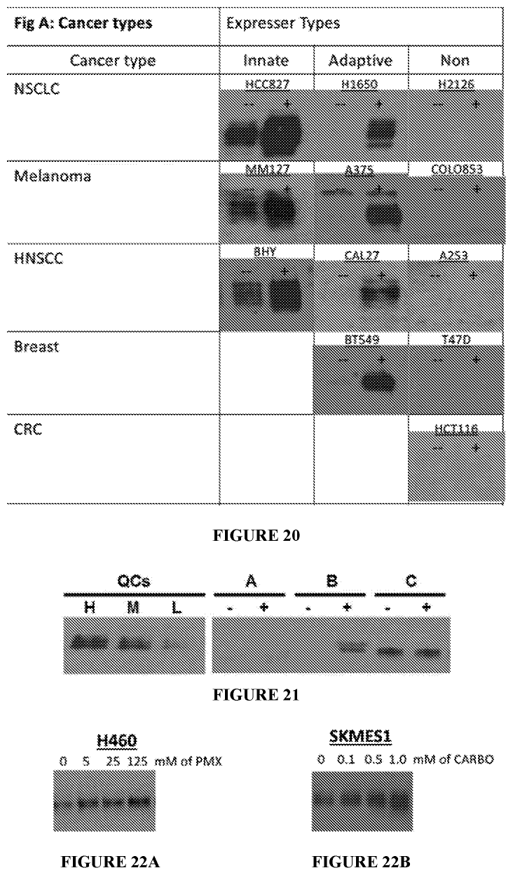

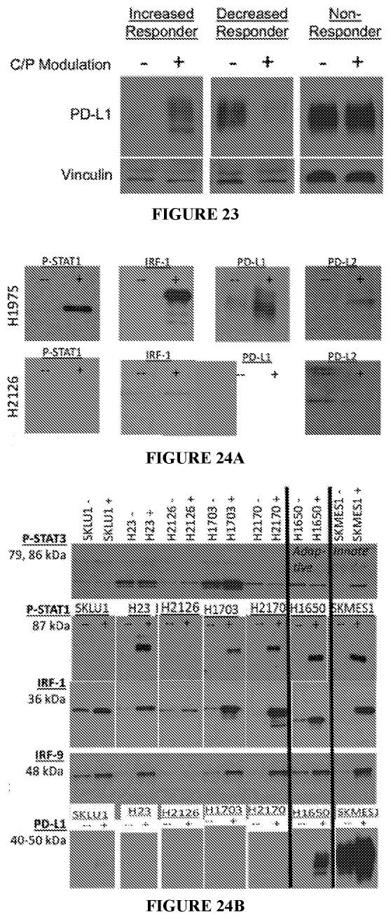

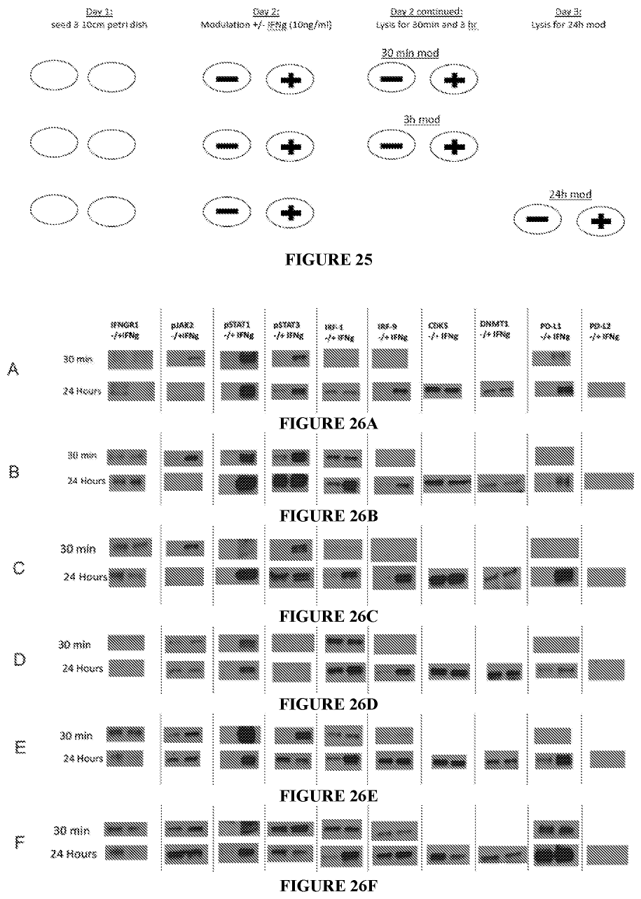

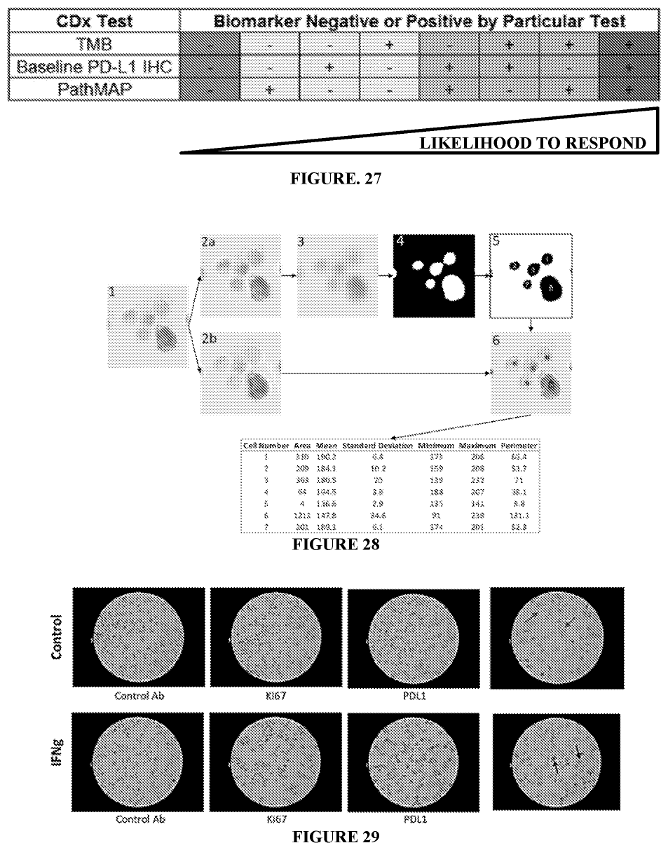

Western Immunoblotting: Samples were separated using Tris-glycine SDS-polyacrylamide gel electrophoresis (Bio-RAD) and transferred to Immobilon-P membranes (Millipore, Mass.). Membranes were blocked for 60 minutes at room temperature in Tris-buffered saline (10 mM Tris-HCl, pH 8.0, 150 mM NaCl) containing 0.05% Tween-20 (TTBS) and BSA (Sigma) and then incubated with BSA in TTBS solution containing primary antibodies for 1 to 3 hours at room temperature (RT) or overnight at 4C. Membranes were washed in TTBS 3×10 minutes prior to secondary antibody for 1 hour at RT. Bound antibodies were visualized with horseradish peroxidase-coupled secondary antibodies and chemiluminescent reagent according to the manufacturer's directions. Antibodies used were as follows: PD-L1 (Abcam, MA), PD-L1 (Cell Signaling) and HRP-anti-rabbit secondary. For p-STAT1 expression, cells were brought into suspension in DMEM+HEPES, no phenol red media and modulated with either vehicle control or 180 units/ml IFN-γ for 30 min in suspension. After 30 min, the cell suspension was lysed directly using 2× complete lysis buffer (Cell Signaling LB+PMSF and protease/phosphatase inhibitors). For IRF-1, PD-L1 and PD-L2, the cells were seeded in vitro and incubated in vitro for 24h in complete RPMI (RPMI supplemented with 10% FBS) to allow for cell attachment. Each sample was then modulated with either vehicle control or 180 units/ml IFNγ for 24 h. After 24h modulation, samples were washed with cold PBS and lysed using 1× complete lysis buffer (Cell Signaling LB+PMSF and protease/phosphatase inhibitors). Sample lysates were clarified and total protein quantified using Pierce 660 nm protein assay. Western blots were run with 5 ug total protein per sample (except for PD-L1 which used 20 ug TP per sample; FIGS. 20-24).

Modulation of PD-L1 by IFNγ: 200,000 cells per sample were seeded and incubated in vitro for 24h in complete RPMI (RPMI supplemented with 10% FBS) to allow for cell attachment before being modulated with either 180 units/ml IFNγ or vehicle control for 24 h. After 24h modulation, samples were washed with cold PBS and lysed using 1× complete lysis buffer (Cell Signaling lysis buffer+PMSF and 1× protease/phosphatase inhibitors). Sample lysates were clarified and total protein quantified using Pierce 660 nm protein assay. Western blots were run as discussed above with 5 ug total protein per sample and primary PD-L1 mAB (cell signaling #13684S) used at 1:2500 concentration, secondary goat-anti-rabbit HRP conjugated (BioRad #STAR124P) at 1:2500 concentration.

Modulation of PD-L1 in NSCLC clinical samples: Three different NSCLC clinical samples were disaggregated and enriched for tumor cells via depletion of red blood cells and immune cells in an automated manner on the SnapPath instruments. Cells had IFNγ (180 units/ml) or vehicle modulation applied also on the SnapPath and then were incubated at 37C on a heat block for 24 hrs before final stabilization in 2× complete lysis buffer (Cell Signaling LB+PMSF and 2× protease/phosphatase inhibitors). Sample lysates were clarified and total protein quantified using Pierce 660 nm protein assay. Western blots were run as discussed above with 5 ug total protein per sample and primary PD-L1 mAB (cell signaling #13684S) used at 1:2500 concentration, secondary goat-anti-rabbit HRP conjugated (BioRad #STAR124P) at 1:2500 concentration.

### Example 2

### Immune Checkpoint Inhibitors Response Prediction Using Image-Based Immunoassays

Immune checkpoint inhibitors response prediction using image-based immunoassays was evaluated in non-small cell lung cancer (NSCLC) cells and melanoma cells.

The human NSCLC cell line SKMES1 was grown in Eagle's Minimum Essential Medium (EMEM; ATCC #30-2003) with 10% Fetal Bovine Serum (FBS) and 1% Penicillin and Streptomycin (P/S). The human NSCLC H460, H1299 and H292 and melanoma cell lines MeWo, HMVII, A375 and CHL1 were grown in RPMI1640 medium with 10% FBS and 1% P/S and 1% sodium pyruvate. Cultured cells were harvested using Accutase, and the cell count and viability were determined using trypan blue dye exclusion. Other methods to collect cells, including scraping or other release can be used.

Between 15,000 to 20,000 cells were put into each chamber of 8-chambered ICC slides in 200 uL medium with or without 10 ng/mL human IFNγ. Cells were incubated for 24h in a 37° C. humidified incubator with 5% CO₂. After treatment, cells were fixed by adding 200 uL of 20% neutral buffered formalin (NBF, Final concentration of NBF at 10%) and incubated for 10 min at room temperature. Cells were washed twice with 1× Phosphate Buffered Saline (PBS) and dried on a slide warmer at 40° C. and stored at −80° C. for short-term storage. To obtain data for proximal biomarkers (e.g. p-STAT-1), incubation times can be lowered to as few as 5 minutes. IFNγ can be readily replaced by other modulators discussed within. Other fixative chemicals can be utilized according the requirement of the type of analytical tools used. Various types of slides can be utilized including those coated to capture and therefore select different cell types. Additionally, the chambers can be coated with extracellular membrane components, such as various types of collagen, elastin, fibronectin, and the alike, to manipulate cell morphology and stiffness and influence PD-L1 expression under varying conditions. Cell lines can be readily switched for other sources of cells, including those isolated from xenografts or clinical samples and can be processed manually or using automated methods (e.g. on SnapPath).

Clinical samples were disaggregated using an automated disaggregation method, on a SnapPath instrument, to maintain viability and cellular integrity. A live cell suspension obtained from SnapPath processed NSCLC clinical tissue samples was fixed for 13 min with 10% NBF. The NBF was then neutralized by diluting with Dulbecco's Modified Eagle Medium (DMEM) and cells were pelleted at 200 g for 5 min. The supernatant was decanted, and the fixed cells were re-suspended in DMEM before seeding onto 8-well chambered slides, dried at 40° C., and stored at −80° C. for short-term storage.

Cells on the chambered slides were stained entirely at RT using the BioGenex RTU Detection System kit components (QD400) unless otherwise indicated. Other detection reagents and methods (e.g. multiplex immunocytochemistry, single of multiplex immunofluorescence, confocal, etc.), including automated staining methods, can be used readily. For staining, slides were rinsed with PBS and endogenous peroxidases were neutralized with “Peroxide Blocking Reagent” for 10 min. Slides were then washed with “Super Sensitive Wash Buffer” before incubation with “Power Block” for 10 min. Next, appropriately diluted primary antibodies for PD-L1 (Cell Signaling #13684S; Clone E1L3N) and appropriate controls are added. Controls utilized included the positive staining control Ki67 (Abcam #ab16667), T-EGFR (Cell Signaling #4267), and Vimentin (Spring Bioscience #M3200) and a negative isotype antibody control (Abcam #ab125938). Ki67, T-EGFR or vimentin are provided as examples of positive controls, but others can be readily used. These controls can be used to normalize signals in conjunction with the negative control isotype antibodies where no staining was observed. Other antibodies for these control proteins can be used readily. Additionally, other antibodies staining nuclear, cytoplasmic, and membrane compartments could be utilized to assist in the determination of the location of PD-L1 or other biomarkers being analyzed such as the nuclear translocation of p-STAT1 from the cytoplasm to the nucleus. Also, other antibodies recognizing other epitopes of PD-L1 to detect various isoforms or variants can be used. After incubation for 30 min, there was a sequential addition and removal of “Super Enhancer Solution” (20 min) “Poly-HRP Reagent” (30 min) and freshly prepared “Diaminobenzidine (DAB) Chromogen” (10 min). Between each step, slides were washed with “RT Super Sensitive Wash Buffer”. Finally, the slide was washed with tap water for 1 min, then “Mayer's Hematoxylin” was applied as a counterstain before being washed with water. Slides were protected using “Super Mount Permanent Mounting Medium” and dried. Staining pattern in sample was assessed manually under microscope using the following scales: 1+=1-25%; 2+=26-50%; 3+=51-75%; and 4+=76-100%; with intensity of staining as 1 for low, 2 for medium, and 3 for high. Other scoring mechanisms can be readily used, including different cut points for normalization scales, normalization from 0-1 or 0-100, or simple dichotomous results such as negative/positive or responder/non-responder.

Images of ICC slides were obtained by using a Canon EOS 6D DSLR camera with a Bayer filter (the detector) connected to a brightfield Olympus BH2 microscope with a halogen lamp and with a 40× objective lens. Images of ICC slides can also be obtained by many other means, including slide scanners, other brightfield microscopes or even confocal microscopes. The detector could be a CMOS camera, a CCD camera, a photodiode, or a photomultiplier tube. The filter is optional, and could be any array filter, a spectral filter, or any band-pass/low-pass/high-pass optical filter. The illumination source can be a halogen lamp, a mercury lamp, a light emitting diode or a laser. A bandpass, lowpass or high pass optical filter can also be used in between the illumination source and the ICC slide. An analog-to-digital converter can be used to convert the analog signal into a digital signal; this can be 8-bit or greater. Images are obtained in a raw intensity format, without any light or color correction.

After obtaining raw images of the ICC slides, the image was analyzed using ImageJ. First, the image was separated into spectral channels corresponding to the Bayer filter: red, green, and blue. Blue and red filters can be replaced with 400-500 nm and 525-625 nm bandpass filters, respectively. The blue (B) and red (R) channels were first inverted. Afterwards, a background subtraction algorithm is applied to both the R and B channel, increasing the lower level (minimum) of brightness until 95% of the non-cellular regions have a brightness level of zero. Next, a blurring filter is applied to the red channel with a blur size of 6. A binary mask is created with the red channel by applying a threshold above the background (brightness=0). This binary mask is zero for non-cellular regions and “one” for cellular regions. The particle analysis algorithm is applied to the binary mask, which creates a set of “regions of interests” for individual cells. For particle size analysis, a minimum size of 5 pixels is used to remove debris and subcellular components. Using the B channel, the median, mean, standard deviation and histogram of pixel intensity was measured for each region of interest (corresponding to one event or cell per region of interest). The region of interest was also used to measure area, perimeter, and circularity for each event. Further normalization of the dataset is performed on the dataset after isotype and unstained controls are measured.

For background subtraction, multiple different methods can be used including frame differencing, mean filter, gaussian average, or temporal average filter. For blurring the image, multiple different methods can be used including a convolution filter or Gaussian smoothing. Thresholding of the image into a binary mask can be performed using multiple methods including algorithms based on the histogram shape or pixel correlation, as well as local methods or entropy-based methods.

FIG. 28 summarizes the steps necessary to process a digital image of cells from an ICC-stained slide. In Step 1, an image is obtained using a microscope and a Canon EOS 6D DSLR camera. In Step 2, the image is separated into a counterstain channel (2a) and a DAB chromogen channel (2b). In Step 3, a gaussian blur is used to filter the counterstain channel, which enables the use of particle tracking algorithms. In Step 4, a thresholding algorithm is used on the image from Step 3 to create a binary mask of noncellular (black) and cellular (white) regions. In Step 5, a particle analysis algorithm is used to create specific “areas of analysis” (AoA) corresponding to each individual cell; each AoA is given an identifying number for data analysis. In Step 6, the area, perimeter, mean pixel intensity and standard deviation of the mean is measured for each AoA using the DAB chromogen channel from Step 2b. This information is provided in tabular format to simplify analysis.

Changes in PD-L1 Expression as Measured Using ICC as a Predictive Test:

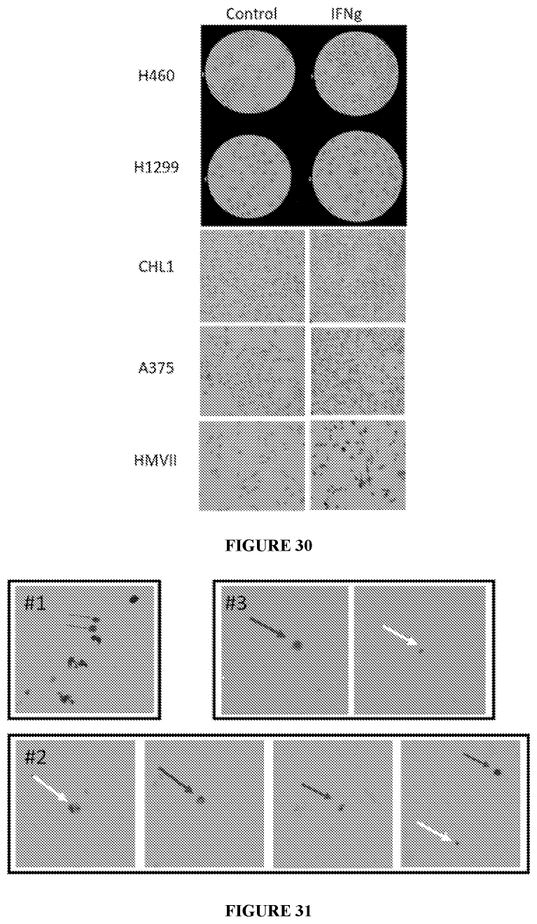

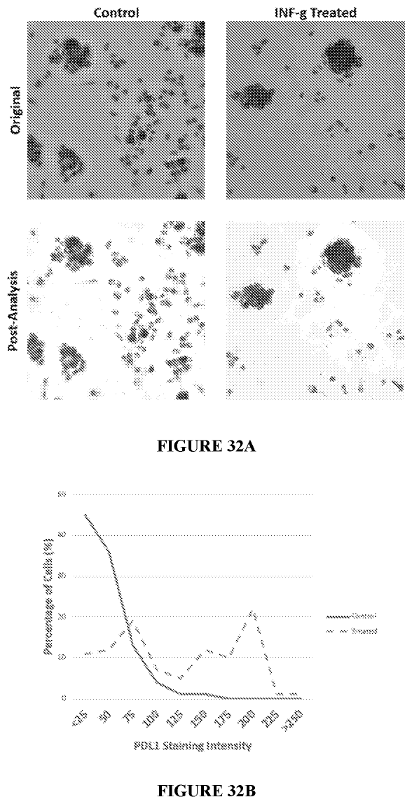

PD-L1 expression was measured in untreated and IFNγ-treated NSCLC and melanoma cell lines as determined by ICC analysis using the E1L3N antibody clone (w). As shown in FIG. 29, NSCLC SKMES1 control cells exhibited PD-L1 staining levels of 4+/1-2 indicating an innate ability to express PD-L1. Upon IFNγ treatment, positivity increased to 4+/3 indicating that this sample has the added ability to adaptively express PD-L1. The PD-L1 staining was both cytoplasmic and membrane using the E1L3N PD-L1 antibody (FIG. 29). Other samples had similar types of innate expression and an adaptive response such as H460 and H292. Other examples of responses demonstrate the utility of functional response to IFNγ to identify samples that are negative at baseline (e.g. H1299, MeWo, HMVII, and CHL1) but have a strong adaptive capacity to express PD-L1 upon exposure to the appropriate stimulator such as IFNγ (FIG. 30; Table 1). For example, a weak adaptive expression was evident for H1299 which had no PD-L1 staining in the control and showed a minor increase in PD-L1 expression in IFNγ treated cells to an intensity of 1+/1-2. Stronger adaptive responses were seen in A375 (1+/3 to 4+/3 upon IFNγ treatment), MeWo (negative to 2+/3), HMVII (negative to 4+/3) and CHL1 (negative to 4+/3) with only a few negative cells in HMVII and CHL1 after treatment with IFNγ (FIG. 30). PD-L1 positive (at varying intensity) and negative tumor-like and immune like-cells were readily observed in NSCLC clinical tissue samples processed through our automated SnapPath instrument (FIG. 31) indicating the approach can be used with complex sample types. In sample #1, strong PD-L1 staining was evident (dark arrows). In sample #2, varying PD-L1 intensities in tumor-like cells were evident including PD-L1 negative (white arrow; far left) and positive (dark arrows). PD-L1 expression in the tumor-like cells varied from low (e.g. middle right) to medium (middle left) to high (far right). Immune-like cells were negative (white arrow; far right) in SnapPath processed NSCLC clinical tissue sample #2. In sample #3, there was PD-L1 positive tumor-like (dark arrow) and negative immune-like cells from SnapPath processed NSCLC clinical tissue. These intensity determinations can be readily automated. For example, as shown in FIG. 32A, images after analysis show a higher contrast than images before analysis. These results are then shown in a qualitative format for the viewer such as quantitative intensity score in a histogram format (FIG. 32B) showing the proportion of cells with DAB intensity ranging from 0 to 255 DAB intensity units for PDL1. In this example, the peak intensity for the treated SKMES1 cell line has a higher intensity than the peak for the control sample.

TABLE 1
Modulation of PD-L1 expression by IFNγ in human
NSCLC and Melanoma Cell Lines: Immunocytochemical
(ICC) analysis as analyzed using anti-PD-L1 antibody
(Clone E1L3N) to residues near the carboxy terminus.

Immunocytochemical Score

| Cell Line | Untreated Control Cells | IFNγ-Treated Cells |
|---|---|---|
| NSCLC | | |
| SKMES1 | 4+/1-2 | 4+/3 |
| H460 | 4+/1-2 | 4+/2-3 |
| H292 | 4+/1 | 4+/3 |
| H1299 | Negative | 1+/1-2 (Few Cells) |
| H1650 | Negative | Negative |
| Melanoma | | |
| A375 | 1+/3 (Few Cells) | 4+/3 |
| MeWo | Negative | 2+/3 |
| HMVII | Negative | 4+/3 |
| CHL1 | Negative | 4+/3 |

Image based immunoassays can be used to detect PD-L1 at baseline and modulated levels to better predict response to immune checkpoint inhibitors than baseline levels alone. Three of four NSCLC (SKMES1, H460 and H292) and one of four melanoma (A375) tested cell lines showed varying patterns of “innate” expression of PD-L1. Notably, these “innate” cell lines responded to IFNγ with increased levels of expression of PD-L1 showing an “adaptive” response. The PD-L1 negative cell lines, “non-expressers” (NSCLC H1299; melanoma cell lines MeWo, HMVII and CHL1) showed an “adaptive” response with an induction of PD-L1 with low to heightened levels upon IFNγ treatment. The detection of PD-L1 positive and negative tumor-like and immune-like cells from three SnapPath processed NSCLC clinical tumor tissue samples was further demonstrated. The data provided demonstration assessing PD-L1 baseline expression and expression capability, in all samples types, including clinical tumor tissue samples to develop companion diagnostics to predict the response to checkpoint inhibitors and other therapies in cancer patients. The quantitative ICC data shown that the intensity of DAB chromogen using automated, digital methods can be successfully detected. The change of intensity by average intensity as well as cell-based histogram methods was successfully quantified. Digital scoring methods allow for a repeatable method of detecting ICC staining without human variation. Higher specificity for DAB chromogen is achieved by optical filtering of transmitted light, making it easier to discern between true DAB staining and blue counterstains.

### Example 3

### Immune Checkpoint Inhibitors Response Prediction Using Flow Cytometry

Samples (150 uL per tube) were first incubated with a blocking reagent at 1:20 for 15 min at room temperature. Alexa 488-conjugated PD-L1 antibody (CST cat #25048, Lot #2) and Alexa 488-conjugated isotype antibody (CST cat #2975, Lot #16) were added to experimental samples and isotype control samples, respectively, at a manufacturer recommended dilution of 1:50 and the samples were incubated for 30 min at room temperature. Other fluorophore-conjugates and PD-L1 antibodies, including those targeting different epitopes, can be readily used. Additionally, antibodies detecting other biomarkers with appropriate processing (e.g. permeabilization of cells) to predict response (e.g. p-STAT-1) can be used. Following the incubation with an antibody, 1 mL of 2% FBS in PBS was added to each tube to dilute the antibody. Samples were then centrifuged at 200 rcf for 5 min. Supernatant was carefully aspirated and 200 uL of 10% neutral buffered formalin (NBF) was added. Samples were then fixed for 10 min at room temperature. NBF was diluted with 1 mL of 2% FBS in PBS and the samples were centrifuged at 200 rcf for 5 min. Supernatant was aspirated and the cells were resuspended in 0.5 mL of 2% FBS in PBS for flow cytometric analysis. In this experiment, 10% NBF was used to fix cells, but other fixatives, for example, 4% paraformaldehyde can be used.

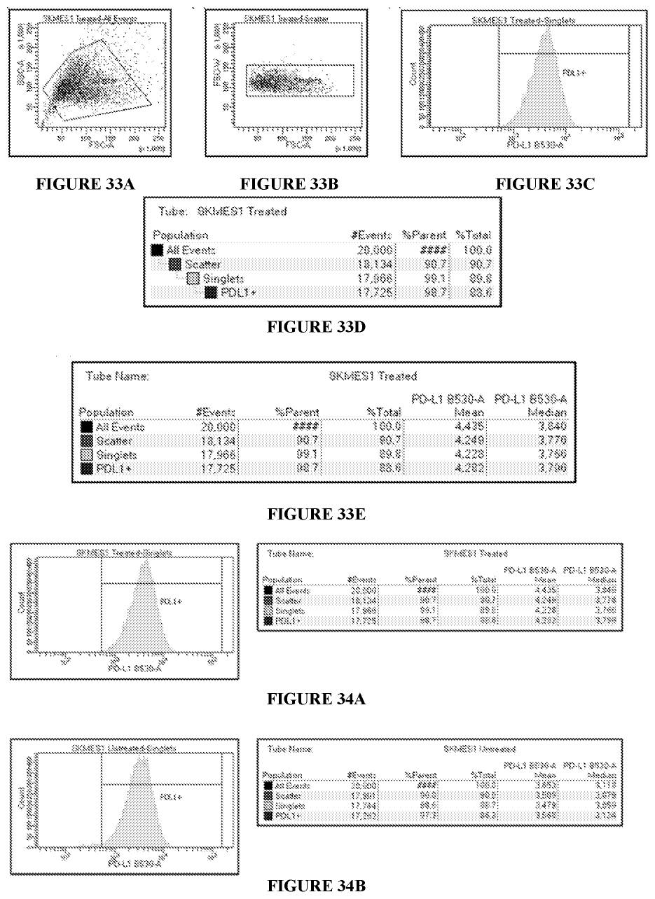

Flow cytometry was performed using BDTM LSR II cytometer and BD FACSDiva™ data acquisition software. Any commercially available flow cytometry analysis software can be used, such as FlowJo (BD) and FCS Express (De Novo Software). Additionally, other flow cytometry instruments can be readily used. A total of 20,000 events were recorded for each sample. A Scatter gate was first applied, and a singlet gate was then applied on the scatter population to exclude doublet population. A 488 nm blue laser and a 530 nm detector were used to capture PD-LL signal. Any signal above the isotype control signal of the singlet population was considered PD-L1+(see FIG. 33). Though it was excluded from this experiment, cell viability can be assessed, and dead cells excluded from analysis, using stains such as Live/Dead™ Fixable Dead Cell Stain (Thermo) for fixed cells, or either 7-AAD (7-Aminoactinomycin D) or Propidium Iodide for unfixed cells.

There are many antibodies against PD-L1 available. As the PD-L1 antibody used in this experiment targeted extracellular portion of PD-L1, samples were not permeabilized. If an antibody which targets intracellular portion is to be used, or if intracellular pools are being assessed, cells can be permeabilized by using commercially available permeabilization reagents or simple detergents such as Triton-X. The ratio of extracellular to intracellular PD-L1 can be used to identify potential false-positive PD-L1 samples where the protein is not sufficiently expressed at the cell surface. In this experiment, PD-L1 was directly detected with a fluorescently-conjugated primary antibody. Alternatively, indirect detection can be performed using unlabeled primary antibody and fluorescently-conjugated secondary antibody. Capabilities of the flow cytometer (lasers and detectors) should be considered when selecting a fluorescent label. As only one color (Alexa 488) was used in this experiment, histogram was used to show the PD-L1 expression data. If multiple colors were to be used in the same experiment, data can be graphed in dot plot, contour plot and density plot formats to determine whether there are double positive and double negative populations.

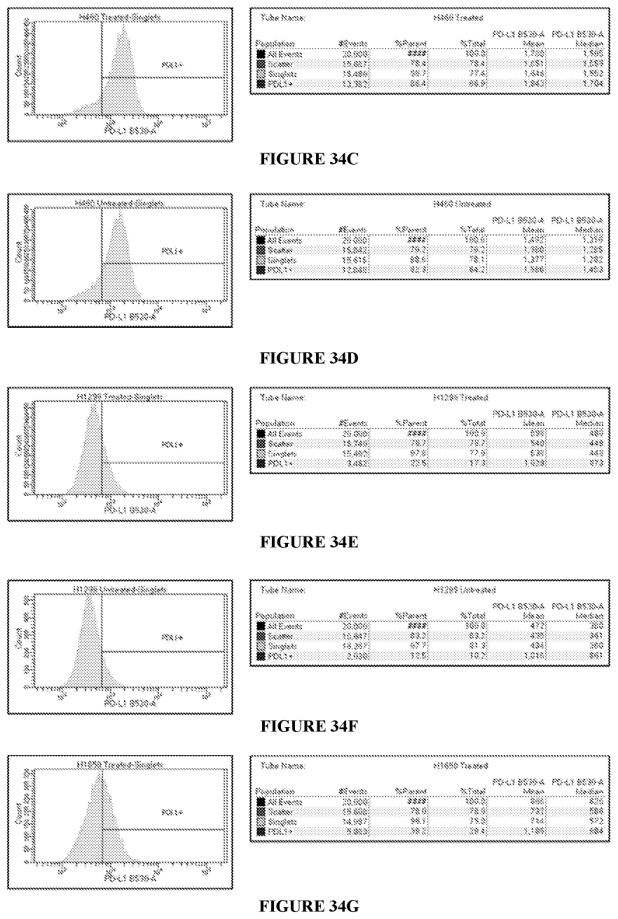

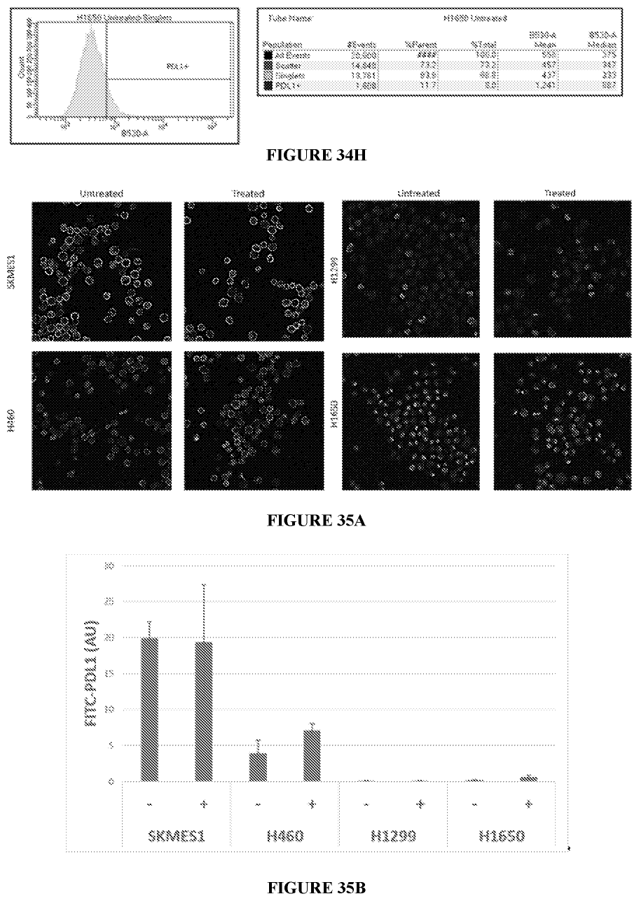

A percent change in PD-L1+ events between Untreated (non-modulated; vehicle control) and Treated (modulated with IFNγ) samples was calculated for each of the four cell lines tested (Table 2). SKMES1 and H460 showed a minimal change in PD-L1+ events upon IFNγ treatment, whereas larger changes were observed in H1299 and H1650. A percent change in PD-L1 signal between Untreated and Treated samples was calculated for each of the four cell lines tested. H1650 showed the highest level of change upon IFN-γ treatment. The mean PD-L1 signal was used to calculate the percent change, however, median (as shown in FIGS. 34A-34H), geometric mean, mode or any other averaging method can also be used. To assess the difference in PD-L1 expression level or number of PD-L1+ events between Treated and Untreated samples, either a percent change or fold change can be calculated. Differences in PD-L1 expression level can also be assessed between a control sample with a known PD-L1 level and a clinical sample with an unknown PD-L1 level. To do so, the assay result of modulated and unmodulated clinical samples should be normalized to that of a control sample. A percent change or fold change between modulated and unmodulated samples can then be calculated using the normalized values. Baseline PD-L1 levels, as well as changes to PD-L1 upon modulation, can be used to predict response to therapy. As mentioned in the Methods section above, the antibody against PD-L1 used in this experiment targets extracellular portion of the PD-L1 molecule and as such, permeabilization was not performed. Antibodies such as those that target other PD-L1 domains would yield different PD-L1 expression patterns depending on the form(s) of PD-L1 present in the cell lines tested. Flow cytometry cannot detect PD-L1 molecules that are already secreted and not bound to other cell surfaces proteins. It is, however, possible to detect various levels of transmembrane and cytoplasmic PD-L1 expression as well as the change in PD-L1 expression upon IFNγ treatment using flow cytometry.

TABLE 2
Number of Events in PD-L1 + population and Mean PD-L1 Signal.

| | | | |
|---|---|---|---|
| | | % change | Fold change |
| Untreated | Treated | (Treated - Untreated)/Untreated | (Treated/Untreated) |

Number of Events in PD-L1 + population

| | | | | |
|---|---|---|---|---|
| SKMES1 | 1726 | 1772 | 3% | 1.03 |
| H460 | 12848 | 13382 | 4% | 1.04 |
| H1299 | 2030 | 3462 | 71% | 1.71 |
| H1650 | 1608 | 5883 | 266% | 3.66 |

Mean PD-L1 signal in Singlets

| | | | | |
|---|---|---|---|---|
| SKMES1 | 3479 | 4228 | 22% | 1.22 |
| H460 | 1377 | 1646 | 20% | 1.20 |
| H1299 | 434 | 538 | 24% | 1.24 |
| H1650 | 437 | 714 | 63% | 1.63 |

The number of events in PD-L1+ population was counted and the mean PD-L1 signal in singlet population calculated using BD FACSDiva software (Data shown in Statistics Tables in FIGS. 34A-34H)

### Example 4

### Immune Checkpoint Inhibitors Response Prediction Using Confocal Microscopy

As another example of imaging/optical based methods to measure PD-L1 expression and predict response to ICC, fluorescence microscopy was used. Fluorescence microscopy includes both epifluorescence and confocal microscopy techniques. As way of example, confocal microscopy was used. In this fluorescence microscopy, light of a specific absorbing wavelength illuminates the specimen, and the fluorescent emission from the specimen is then captured by the objective lens where it is projected onto a detector. In confocal microscopy, the use of a pin hole and special detectors are used to block out of focus light, allowing for the imaging of subcellular components of the specimen.

To obtain fluorescent specimens, similar procedures are used when compared to flow cytometry. The sample is stained with a primary antibody that is specific for a protein of interest. The primary antibody can be conjugated to a fluorescent molecule or a secondary antibody can be used that attaches to the primary antibody. For intracellular staining of cells, the specimen can be fixed and permeabilized using methanol, formalin or a fixative containing a detergent. Subsequently, the specimen is stained with primary and/or secondary antibodies. For this dataset, samples were stained for PD-L1 in suspension using the same protocol that was used for the flow cytometry dataset. The protocol used was as follows:

Samples (150 uL per tube) were first incubated with a blocking reagent (BD cat #564219, Lot #9305316) at 1:20 for 15 min at room temperature. Alexa 488-conjugated PD-L1 antibody (CST cat #25048, Lot #2) and Alexa 488-conjugated isotype antibody (CST cat #2975, Lot #16) were added to experimental samples and isotype control samples, respectively, at a manufacturer recommended dilution of 1:50 and the samples were incubated for 30 min at room temperature. Other fluorophore-conjugates and PD-L1 antibodies, including those targeting different epitopes, can be readily used. Additionally, antibodies detecting other biomarkers with appropriate processing (e.g. permeabilization of cells) to predict response (e.g. p-STAT-1) can be used. Following the incubation with an antibody, 1 mL of 2% FBS in PBS was added to each tube to dilute the antibody. Samples were then centrifuged at 200 rcf for 5 min. Supernatant was carefully aspirated and 200 uL of 10% neutral buffered formalin (NBF) was added. Samples were then fixed for 10 min at room temperature. NBF was diluted with 1 mL of 2% FBS in PBS and the samples were centrifuged at 200 rcf for 5 min. Supernatant was aspirated and the cells were resuspended in 0.5 mL of 2% FBS in PBS for flow cytometric analysis. In this experiment, 10% NBF was used to fix cells, but other fixatives, for example, 4% paraformaldehyde can be used. For localization experiments an anti-phosphotyrosine antibody, anti-human/mouse, PE-Vio® 770 (Miltenyi Biotec, Cat #130-109-112) and an anti-PD-L1 antibody, anti-human, Brilliant Violet 650 (BV650; BD Biosciences, Cat #563740) were used. After staining, a final concentration of 1 ug/mL of Hoechst 33342 was added to the cell suspension to stain the nuclei of the cell. After incubating the sample for 10 minutes, 10 uL of the cell suspension was added to a microfluidic chip for imaging on a Leica SPX5 confocal microscope. A 40× oil immersion objective lens was used. A 405 nm diode laser was used to excite the Hoechst 33342 molecules while a 488 nm laser was used to excite the FITC molecules. A transmitted differential interference contrast channel was used to detect the brightfield of the sample.

PD-L1 was assessed in four cell lines (FIG. 35A). PD-L1 was quantified by analyzing FITC-PD-L1 using an 8-bit photomultiplier tube and quantified as raw arbitrary units (FIG. 35B). The PD-L1 expression of the four cell lines was quantified in treated and untreated conditions. SKMES1 showed innate expression of PD-L1 whereas H460 showed adaptive expression. H1299 and H1650 were both negative for PD-L1 in untreated and IFNγ treated samples. Thus, PD-L1 expression can be measured qualitatively and quantitatively using confocal microscopy and other fluorescent microscopy techniques.

### Example 5

### Immune Checkpoint Inhibitors Response Prediction Using PD-L1 mRNA Expression

Comparable to measuring PD-L1 with various immunoassays, the abundance of PD-L1 mRNA levels, or other more proximal gene expression biomarkers (e.g. IRF1) can be measured to predict response to various ICC therapies. mRNA can be measured by real-time PCR, conventional RT-PCR or northern blotting. Additionally, the presence, abundance and ratio of different PD-L1 mRNA forms can be used to predict response. As way of example, PD-L1 mRNA were measured using primers amplifying within exon 3-4 (extracellular region of the transcribed protein that interacts with PD1). Other amplicons detecting the same or other forms can be used readily. For example, amplicons corresponding to the transmembrane and cytoplasmic tail to confirm full length plasma membrane bound protein is transcribed, or regions overlapping with either PD1 or anti-PD-L1 binding sites. Five cell lines (H460, H1299, H1650, H2126 and SKMES1) were treated with vehicle or 10 ng/mL IFNγ for 24h. More complex sample types, including cells isolated from solid tumors from preclinical and clinical cancers be used. Other time points, including those as early as 1h for IRF or 4h for PD-L1, can be readily used. RNA was isolated from the cells using an NEB kit (Cat #T2010) per the manufacturer's instructions. Isolated RNA was estimated using a NanoDrop nanometer and checked for its quality by checking the 260/280 ratio. RNA concentration was brought to 40 ng/uL in all the samples to take equal quantity for further steps of RT and real time PCR. One step RT-qPCR was performed using a Power SYBR Green RNA to Ct One step kit (Cat #4931198) as per the manufacturer's instructions. A 200 ng RNA template was taken for total 20 uL reaction per well in 96 well PCR microplate for Roche 480 Light cycler. The following primer pairs (Sigma) were used for PD-L1 and GAPDH and were used at a concentration in the reaction mixture of 400 nM:

(SEQ ID NO: 1)
PD-L1 Forward Primer 5′ ATGCCCCATACAACAAAATC 3′
(SEQ ID NO: 2)
PD-L1 Reverse Primer 5′ GACATGTCAGTTCATGTTCAG 3′
(SEQ ID NO: 3)
GAPDH Forward Primer 5′ CTTTTGCGTCGCCAG 3′
(SEQ ID NO: 4)
GAPDH Reverse Primer 5′ TTGATGGCAACAATATCCAC 3′

A Roche 480 Light cycler was used for performing the one step real time PCR. Data was analyzed using 2\hat{ }-ΔΔCt method normalized by GAPDH for relative levels. Alternative housekeeper genes may be readily used.

The expression of PD-L1 mRNA in untreated and interferon gamma (IFN gamma) treated cell lines was detected. H1650 and H1299 cell lines showed a relative increase of PD-L1 mRNA upon IFNγ exposure, at 2.2 fold and 1.7 fold increase, respectively. In comparison, the H460 and H2126 cell lines showed a decrease in mRNA levels and SKMES1 showed no change in expression with the treatment. This change is consistent with other detection methods described herein, demonstrating the ability to utilize the method to predict response to therapy.

### Example 6

### Modulation and Measurement of Pd-L1 Expression by Enhancer Molecules and Therapies

Cell lines were acquired from ATCC, Sigma, or DSMZ. Human non-small cell lung carcinoma (NSCLC) cell lines H292 (ATCC no. CRL-1848.), H1299 (ATCC no. CRL-5803), H520 (ATCC no. CRL-5811), CALU6 (ATCC no. HTB-56), H1975 (ATCC no. CRL-5908), H460 (ATCC no. HTB-177), H2126 (ATCC no. CCL-256), H1650 (ATCC no. CRL-5883), and H2087 (ATCC no. CRL-5922) were used for this experiment along with head and neck squamous cell carcinoma (HNSCC) cell lines BHY (DSMZ ACC 404), CAL27 (ATCC no. CRL-2095), KYSE410 (Sigma), and A253 (ATCC no. HTB-41), melanoma cell lines A375 (ATCC no. CRL-1619), COL0853 (Sigma), and MM127 (Sigma) pancreatic cancer cell lines BXPC3 (ATCC no. CRL-1687), colorectal cancer cell lines HTB38 (ATCC no. HT-29), HCT116 (ATCC no. CCL-247EMT), and breast cancer cell lines BT549 (ATCC no. HTB-122), BT20 (ATCC no. HTB-19), and T47D (ATCC no. HTB-133). Cells were maintained in manufacturer-recommended conditions at 37° C. inside humidified 5% CO2 incubators. Sub-cultivation and harvesting for all cell lines were performed using Accutase (GIBCO Life Technologies; cat. no. A1110501) at 37° C. for 10 minutes. Cell viability was measured using trypan blue exclusion staining in conjunction with the Countess II Automated Cell Counter (Thermo Fischer Scientific; cat. no. AMQAX1000). Measurements were made in duplicate and the average number of viable cells was recorded prior to experiments.

Enhancer Molecule and Therapies: IFNγ, (10 ng/ml; Sigma Aldrich, cat.no 11040596001), TNFα (10 ng/ml; BD Biosciences cat. no. 554618), Osimertinib (100 ng/ml; Selleckchem cat.no. S7297), Everolimus (100 ng/ml; Selleckchem cat.no S1120), GSK458 (10 ng/ml; Selleckchem cat. no. S2658), TNFb (50 ng/ml; Sigma Aldrich cat. no.), GSK795 (1,000 ng/ml; Selleckchem cat. no. S7492), IL-12 (50 ng/ml; Sigma Aldrich SRP3073), GM-CSF (50 ng/ml; Sigma Aldrich cat. no. G5035), IL-1a (1 ng/ml; Sigma Aldrich cat. no. 12778-), IL-6 (50 ng/ml; Sigma Aldrich), IL-17 (100 ng/ml; Sigma Aldrich cat. no. SRP3080), TGFb (1 ng/ml; Sigma Aldrich cat. no. T7039), and IL-10 (50 ng/ml; Sigma Aldrich cat. no. SRP3071) were administered to cell lines for various times including 30 minutes, 3 hour, and 24 hour time intervals prior to cell lysis and protein extraction.

To determine the effects of the aforementioned molecules on the modulation of PD-L1, we selected various NSCLC, HNSCC, breast cancer, melanoma, colorectal cancer, and pancreatic cancer cell lines as examples. Other diseases or cell lines could be used readily as could more complex samples such as cells isolated from xenografts and clinical samples. Cells were plated, for three different time points (30 minutes, 3 hours, and 24 hours), inside 6-well petri dishes with 2 mL of appropriate medium. Plates were incubated for 24 hours in 5% CO2 incubators at 37° C. After 24 hours, each 6-well plate was washed with PBS (Gibco Life Technologies, cat. no. 10010023) followed by fresh media being added along with each cell line's vehicle control or appropriate modulator for each specific time point, and were placed in CO2 incubators until their modulation time point was reached. Other conditions can be readily used, such as processing on automated systems such as SnapPath. At the end of each time point, while keeping plates on ice, cell lysis was conducted by aspirating culture medium from each plate, washing of each plate three times with 5 ml of cold PBS (Gibco Life Technologies, cat. no. 10010023) and culminated with the addition of cold 1× lysis buffer containing (Cell Signaling, cat. no. 9803 S) protease inhibitor cocktail (Cell Signaling, cat. no. 5871S), phosphatase inhibitor cocktail (Cell Signaling, cat. no. 5870S), and PMSF (Fisher Scientific, cat. no. ICN19538101). Plates were then placed on ice for ten minutes, swirling each plate intermittently. Aliquots of protein lysates were prepared and then stored at −80° C. until protein quantification and Western blot analysis was to be conducted. The protein concentration of each lysate was analyzed by using the Pierce 660 Protein Assay Reagent (Thermo Fisher Scientific, cat. no. 22660) and testing of each sample was conducted in duplicate. Two μg of protein was then loaded per lane into 10% Midi-PROTEAN TGX Precast Gels (Bio Rad, cat. no. 5671034) along with Kaleidoscope Prestained Protein Standard (BioRad, cat. no. 1610375) which ran at 220V for 45 minutes. Proteins were transferred to mini-PVDF membranes (BioRad, cat. no. 1704156) in 3 minutes using the Trans-Blot Turbo Transfer System (BioRad, cat. no. 1704150EDU). Each membrane was immediately blocked in 5% non-fat dry milk (Signature Kitchens) in TBST (10×TBS, BioRad, cat. no. 1706435 and Tween 20, BioRad, cat. no. 170-6531; pH 7.4) for one hour followed by 3×10 minutes washes in TBST. Individual membranes were incubated at 4° C. for 24 hours with primary antibody (in 2% BSA) for PD-L1 (1:2,500 dilution; Cell Signaling, cat. no. 13684) and GAPDH (1:5,000 dilution; Cell Signaling, cat. no.) with each antibody being diluted in 2% BSA (Sigma Aldrich, cat. no. A9647-100G).

Following their 24-hour incubation, membranes were washed with TBST followed by probing with goat anti-rabbit HRP-conjugated secondary antibody in 5% milk (1:5,000 dilution; BioRad, cat. no. 1706515) for one hour at room temperature followed by 3×10 minutes washes with TBST. Membranes were then briefly rinsed with deionized water followed by incubation for five minutes in Clarity Western ECL Substrate (BioRad, cat. no. 1705061). Exposure of each membrane to autoradiography film (GenHunter, cat. no. 581) was conducted for enhanced chemiluminescent detection. GAPDH was analyzed to confirm equal loading of samples (example lanes shown). This or other housekeeper proteins can be used to normalize signals using densitometry. Quality control samples (example lanes shown) were run on each gel, with known amounts of PD-L1 equaling “low”, “medium”, and “high”. These samples, and/or standard curve samples (e.g. a blank sample spiked with a known amount of PD-L1 and then serially diluted 2-fold with additional blank sample), can be used to confirm the assay worked successfully and to quantify the abundance of PD-L1 in samples. The abundance of PD-L1 can be readily measured using other forms of immunoassay, such as dot blot, ELISA, and Luminex using appropriate antibodies and reagents.

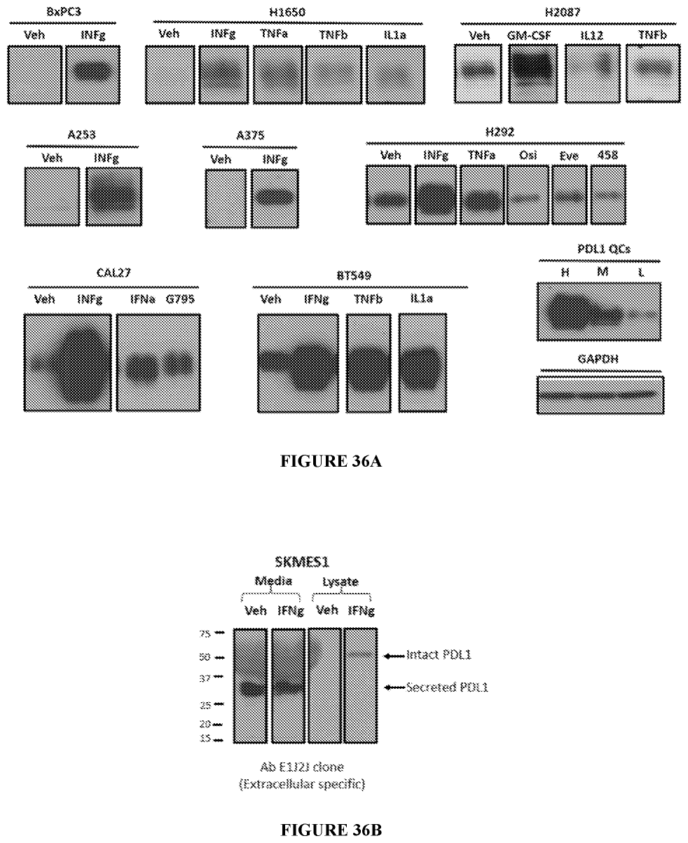

Modulation of samples resulted in various types of responses from sample to sample (FIG. 36A and Table 3) demonstrating the potential utility of modulators when used in the correct context and with appropriate predictive testing. For example, TNFb, GSK458, everolimus, and osimertinib could elicit both up and down regulation of PD-L1, depending on the sample. In contrast, IFNα, IFNγ, TNFα, TGFb, IL1α, and GMCSF only led to upregulation of PD-L1 but not in all samples.

TABLE 3
Context Dependent Responses to EM and MTAs

| Cancer | Cell Line | Modulator | PD-L1 Response |
|---|---|---|---|
| NSCLC | H460 | IFNγ, TNFa | Up |
| | | Others | None |
| | H2126 | IL1a | Up |
| | | Others | None |
| | H1650 | IFNa, IFNγ, TNFa, TNFb, IL1a | Up |
| | | Others | None |
| | H2087 | IFNγ, GM-CSF | Up |
| | | TNFb | Down |
| | | Others | None |
| | Calu6 | IFNγ | Up |
| | | Others | None |
| | H292 | IFNγ, TNFa, IL1a | Up |
| | | Osimertinib, everolimus, GSK458 | Down |
| | | Others | None |
| | H1299 | IFNγ | Up |
| | | Others | None |
| | H1975 | IFNa, IFNγ, TNFb, IL1a | Up |
| | | GSK795, IL12 | Down |
| | | Others | None |
| | H520 | IFNγ | Up |
| | | Others | None |
| Head and neck | A253 | IFNγ | Up |
| | | Others | None |
| | CAL27 | IFNa, IFNγ, GSK795 | Up |
| | | Others | None |
| | BHY | IFNγ, Osimertinib, GSK795 | Up |
| | | Others | None |
| | KYSE410 | IFNa, IFNγ, Osimertinib, IL1a | Up |
| | | Others | None |
| Melanoma | A375 | IFNγ | Up |
| Pancreatic | BxPC3 | IFNa, IFNγ, everolimus, GSK458 | Up |
| Colorectal | HCT116 | All | None |
| | HTB38 | All | None |
| Breast | T47D | All | None |
| | BT549 | IFNa, IFNγ, TNFa, IL1a, GSK795 | Up |
| | | Others | None |
| | BT20 | IFNa, IFNγ, GSK458, TNFb, IL1a | Up |
| | | Others | None |

Different forms of PDL1 can be expressed at baseline as well as after modulation, and the abundance of each form will impact response to therapy. The two major forms are membrane bound and soluble (secreted or intracellular compartments). The relative ratios of the forms, and their cellular locations, can be changed by a modulator and will require different strategies for optimal ICI therapy. As way of example, the cell line SKMES1 was treated with vehicle or 10 ng/mL IFNg for 24 h. After treatment, media was collected and concentrated using an Amicon ultra 0.5 mL centrifugal filter with a 10 kDa cutoff. The retentate, containing proteins >10 kDa, was recovered in a clean tube at 14000 rpm for 5 min. Simultaneously, lysates of the same cells were made. PDL1 was then detected using 10% Tris-Glycine SDS gels and PVDF membranes. The western blot was carried out using 5% non-fat dry milk in TBST for 1h, followed by incubation with primary antibody against PDL1 (clone E1J2J; raised to the extracellular portion of PDL1) in 2% BSA for overnight, then anti-rabbit HRP secondary antibody before using a chemiluminescent reagent (Bio-Rad). We detected the expression of secreted and intact PDL1 in both vehicle and IFNg-treated SKMES1 cell lines (FIG. 36B). Thus, by using westerns where the intact and secreted forms of PDL1 are first resolved by MW and different types of sample/resource material (media vs lysate or blood/plasma vs tumor), the response to therapy can be predicted. Other means can be used, for instance using immunoassays with antibodies that recognize the specific forms or functionality of PDL1. Detection of secreted forms of PDL1, indicates patients would respond better to anti-PD1 therapeutic antibodies that target PD1 on immune cells, rather than anti-PDL1 antibodies due to the secretion of PD-L1 which would act as a decoy in place of PDL1 located on tumor cell surfaces.

Although the invention has been described with reference to the above example, it will be understood that modifications and variations are encompassed within the spirit and scope of the invention. Accordingly, the invention is limited only by the following claims.

1. A method to alter the level, form, and localization of immune checkpoint proteins using a modulator in a biological sample or subject to produce a more robust response to immune checkpoint protein inhibitor (ICI) and/or to identify, monitor, prognose, and predict responsiveness to an ICI monotherapy regime and/or combination regimes comprising:
a) exposing a biological sample or subject which would otherwise not optimally respond to immune checkpoint inhibitors to the modulator to produce a robust response; and/or
b) measuring static biomarker(s) and/or determining the difference between dynamic biomarker(s) in an unmodulated portion of the sample and after contacting one or more portions of the sample with one or more modulators ex vivo, prior to, during, simultaneously with, throughout, or following the administration of ICI monotherapies and/or combination therapies; wherein the difference in the basal and modulated biomarker(s) and the modulated portion(s) of the sample is expressed as a value which is predictive of a positive or negative response to ICI monotherapies and/or combination therapies.

2. The method of claim 1, wherein the sample is from a subject with cancer.

3. The method of claim 2, wherein the cancer is selected from the group consisting of colorectal, esophageal, stomach, lung, mesothelioma, prostate, uterine, breast, skin, endocrine, melanoma, urinary, pancreas, ovarian, cervical, head and neck, liver, bone, biliary tract, small intestine, hematopoietic/blood cancers (myeloma, leukemia, and lymphoma), vaginal, testicular, anal, kidney, brain, eye cancer, leukemia, lymphoma, soft tissue, melanoma, mixed types, and metastases thereof.

4. The method of claim 1, wherein the sample is from single or multiple tumor tissue of an unknown primary or any type of tumor.

5. The method of claim 3, wherein the tumor sample is from a solid tumor.

6. The method of claim 5, wherein the tumor sample is obtained by fine needle aspiration, core biopsy, collecting circulating tumor cells, surgical excision, or other tumor sample acquisition method.

7. The method of claim 1, further comprising stratification of patients on their predicted responsiveness to a therapeutic agent or therapeutic regimen and providing a positive or negative treatment value which corresponds to positive or negative clinical outcome, respectively.

8. The method of claim 1, further used to identify, monitor, predict the existence of and characterize functional sub-populations of cancer cells, which can be used to predict a positive or negative response to ICI monotherapies and/or combination therapies.

9. The method of claim 8, wherein the identification and characterization of functional sub-populations can be used to predict and identify the mechanisms of innate and acquired resistance to ICI monotherapies and/or combination therapies.

10. The method of claim 1, wherein the biological sample is a cancer cell or a cancer cell subpopulation, wherein the cancer cell or cancer cell subpopulation comprises a cancer stem cell that expresses one or more of CD133, CD44, ABCG2, and/or ALDH1A1 and/or that does not stain with Hoechst.

11. (canceled)

12. The method of claim 1, wherein the modulator is a biologic agent, biosimilar, derivative, mutant, peptide, fragment, analog, or mimetic of the modulator.

13. The method of claim 12, wherein the modulator is an interferon (IFN).

14. The method of claim 13, wherein the interferon is IFN-α, IFN-β, or IFN-γ.

15-16. (canceled)

17. The method of claim 12, wherein the modulator is fibroblast growth factor (FGF), epidermal growth factor (EGF), vascular endothelial growth factor (VEGF), prostaglandin E2 (PGE2), prostacyclin (PGI2), prostaglandin D2 (PGD2) and prostaglandin F2α (PGF2α), indoleamine-pyrrole 2,3-dioxygenase (IDO), granulocyte-macrophage colony-stimulating factor (GM-CSF), tumor necrosis factor alpha (TNFα), tumor growth factor beta (TGFβ), a derivative thereof, a mutant thereof, a peptide thereof, a fragment thereof, an analog thereof or a mimetic thereof.

18-82. (canceled)

83. The method of claim 1, wherein the biomarker is the localization, level, and/or state of a molecule or molecules.

84. The method of claim 83, wherein the molecule(s) being measured is a protein or a nucleic acid.

85. The method of claim 83, wherein the state of the molecule(s) being measured is phosphorylation, acylation, alkylation, amidation, glypiation, glycation, glycosylation, ubiquitination, degradation product(s), truncation, mutation status, or binding of the molecule(s) to promoters that induce programmed cell death protein 1 (PD-1) ligand expression including Gamma Activated Sequence (GAS) promoter/enhancer regions and the promoter of PD-1 ligand genes, methylation status, chromatin modification of the promoter/enhancer region, and the status of the SWI/SNF complexes.

86. The method of claim 83, wherein the localization of the molecule(s) being measured is extracellular or cellular, wherein cellular localization comprises intracellular, compartmentalized, nuclear or nucleoli, and membrane bound; wherein compartmentalized localization includes Golgi, endoplasmic reticulum, lysosomal, endosomal, exosomal, mitochondrial, vacuole, and cytosolic localization; and wherein membrane bound includes plasma, nuclear and other organelle membranes.

87. The method of claim 83, wherein the molecule(s) being measured is PD-1, PD-1 ligand (PD-L1), PD-L2, AZAR, CD40, CD40L, galectin-9, IDO1, CTLA-4, KIRs, 4-1BB, GITR, T cell immunoglobulin mucin 3 (TIM-3), LAG-3 (CD223), Killer immunoglobulin-like receptors (KIRs), VISTA, Adenosine, MEW I and/or MEW II.

88. The method of claim 83, wherein the molecule(s) being measured is in a pathway that influences PD-1 ligand expression including the immediate target or downstream target of an Interleukin; is FGF, EGF, VEGF, PGE2, PGI2, PGD2, PGF2α, IDO, or GM-CSF; is in the TNF-α, the TGF-β, the IFNα, IFNβ, or IFNγ response pathways, in the JAK/STAT pathway, in the EGFR response pathway, in the PI3K pathway, in the MAPK pathway, and/or in the mevalonate pathway.

89. The method of claim 88, wherein the molecule(s) being measured is IFN-alpha, beta and/or gamma receptors.

90. The method of claim 88, wherein the molecule(s) being measured is from the STAT family.

91. The method of claim 90, wherein the STAT family member is STAT-1, STAT-2, STAT-3 or STAT-4.

92. The method of claim 88, wherein the molecule(s) being measured is from the Janus Kinase (JAK) family.

93. The method of claim 92, wherein the JAK family member is JAK-1 or JAK-2.

94. The method of claim 88, wherein the wherein the molecule(s) being measured is from the Interferon Regulatory Factors (IRF) family.

95. The method of claim 94, wherein the molecule measured is IRF-1 or IRF-9.

96-121. (canceled)

122. The method of claim 1, wherein the biomarker(s) are measured using immunoassays, multiplexed assays, PCR, transcription factor assays, DNA hypersensitivity assays, nucleic acid or sequencing/mutation testing.

123. The method of claim 122, wherein the immunoassay is selected from the group consisting of a western blot, dot blot, ELISA, immunohistochemistry, immunocytochemistry, and immunofluorescence.

124. The method of claim 122, wherein the multiplexed assay is selected from the group consisting of flow cytometry, microarrays, and bead-based including Luminex multiplex assays.

125. The method of claim 122, wherein the PCR is selected from the group consisting of qPCR, RT-PCR, real-time PCR and endpoint PCR.

126. The method of claim 122, wherein the transcription factor identification assay is selected from the group consisting of protein arrays, chromatin immunoprecipitation (CHIP) and CHIP-seq assays, DNA precipitation and DIP-seq assays, microsphere assays, DNase sensitivity and gel shift assays.

127. The method of claim 122, wherein the nucleic acid or sequencing mutation testing is selected from the group consisting of massively parallel signature sequencing (MPSS), 454 pyrosequencing, Illumina (Solexa) sequencing, SOLiD sequencing, Ion Torrent semiconductor sequencing, Heliscope single molecule sequencing, single molecule real-time (SMRT) sequencing, sequencing by hybridization, and sequencing with mass spectrometry.

128. The method of claim 1, wherein the biological sample is processed in part, or entirely, using one or more manual methods and/or automated systems.

129-138. (canceled)
